SEQUENCE LISTING (34 sequences)

SEQ ID NO: 1 - DNA, 19 bp, synthetic construct
  source 1..19: mol_type = other DNA
ggggttctgagaggaaggt

SEQ ID NO: 2 - DNA, 20 bp, synthetic construct
  source 1..20: mol_type = other DNA
ccgtcatccttcacgctact

SEQ ID NO: 3 - DNA, 27 bp, synthetic construct
  source 1..27: mol_type = other DNA
caatattcctcactgctgcctcccgta

SEQ ID NO: 4 - DNA, 20 bp, synthetic construct
  source 1..20: mol_type = other DNA
tgcgggtatcgaacaggatt

SEQ ID NO: 5 - DNA, 20 bp, synthetic construct
  source 1..20: mol_type = other DNA
ggtaaggttcctcgcgtatc

SEQ ID NO: 6 - DNA, 41 bp, synthetic construct
  source 1..41: mol_type = other DNA
ttaacgctttcgcttggccacagtagtccgcacggtaaacg

SEQ ID NO: 7 - DNA, 39 bp, synthetic construct
  source 1..39: mol_type = other DNA
gtacgccggcaacggtgaaaacatgttcctccgcttgtg

SEQ ID NO: 8 - DNA, 17 bp, synthetic construct
  source 1..17: mol_type = other DNA
ggccgaacagcgagcat

SEQ ID NO: 9 - DNA, 19 bp, synthetic construct
  source 1..19: mol_type = other DNA
caaaggaattgacgggggc

SEQ ID NO: 10 - DNA, 19 bp, synthetic construct
  source 1..19: mol_type = other DNA
gatggggatgcgttccatt

SEQ ID NO: 11 - DNA, 20 bp, synthetic construct
  source 1..20: mol_type = other DNA
ccatagggcagtcatccttc

SEQ ID NO: 12 - DNA, 39 bp, synthetic construct
  source 1..39: mol_type = other DNA
ccaatgtgggggaccttcctcggtaacggcccacctagt

SEQ ID NO: 13 - DNA, 40 bp, synthetic construct
  source 1..40: mol_type = other DNA
cacggtccaaactcctacggggctacttggctggttcagg

SEQ ID NO: 14 - DNA, 19 bp, synthetic construct
  source 1..19: mol_type = other DNA
tgctacctatccccaagac

SEQ ID NO: 15 - DNA, 21 bp, synthetic construct
  source 1..21: mol_type = other DNA
aggaatattggtcaatgggcg

SEQ ID NO: 16 - DNA, 20 bp, synthetic construct
  source 1..20: mol_type = other DNA
aggatgaaggtcctacggat

SEQ ID NO: 17 - DNA, 19 bp, synthetic construct
  source 1..19: mol_type = other DNA
agccccaaactttcacagc

SEQ ID NO: 18 - DNA, 39 bp, synthetic construct
  source 1..39: mol_type = other DNA
ggcacggagttagccgatgcgggaataaaccctcccacg

SEQ ID NO: 19 - DNA, 38 bp, synthetic construct
  source 1..38: mol_type = other DNA
gcagccgcggtaatacggagcttaacgacccgtctgcg

SEQ ID NO: 20 - DNA, 25 bp, synthetic construct
  source 1..25: mol_type = other DNA
tcataaggtacatacaagctcccac

SEQ ID NO: 21 - DNA, 24 bp, synthetic construct
  source 1..24: mol_type = other DNA
ccggatttattgggtttaaaggga

SEQ ID NO: 22 - DNA, 27 bp, synthetic construct
  source 1..27: mol_type = other DNA
  misc_feature 9..10: note = linked to quencher molecule ZEN (Zero-Mode Waveguide Enhanced Non-Fluorescent Quencher)
  misc_feature 1: note = labeled with FAM (6-carboxyluorescein, a fluorescent dye)
  misc_feature 27: note = linked to quencher molecule IABkFQ (Internal Abasic Site Cleaving Quencher)
caatattcctcactgctgcctcccgta

SEQ ID NO: 23 - DNA, 21 bp, synthetic construct
  source 1..21: mol_type = other DNA
cggccaaatactcctgatcgt

SEQ ID NO: 24 - DNA, 24 bp, synthetic construct
  source 1..24: mol_type = other DNA
gcttgttgcgttccttgagataat

SEQ ID NO: 25 - DNA, 36 bp, synthetic construct
  source 1..36: mol_type = other DNA
  misc_feature 1: note = LABELED WITH FAM (fluorescent dye 6-carboxyfluorescein)
  misc_feature 9..10: note = linked to ZEN (Zero-Mode Waveguide Enhanced Non-Fluorescent Quencher) quencher molecule
  misc_feature 36: note = linked to IABkFQ (Internal Abasic Site Cleaving Quencher) quencher molecule
aggcacctatgtcctttacctcatcaactacagaca

SEQ ID NO: 26 - DNA, 27 bp, synthetic construct
  source 1..27: mol_type = other DNA
gcatgaatttagcttgctaaatttgat

SEQ ID NO: 27 - DNA, 22 bp, synthetic construct
  source 1..22: mol_type = other DNA
acctcatacggtattaatccgc

SEQ ID NO: 28 - DNA, 14 bp, synthetic construct
  source 1..14: mol_type = other DNA
  misc_feature 1: note = labeled with FAM (6-carboxyluorescein, a fluorescent dye)
  misc_feature 9..10: note = linked to quencher molecule ZEN (Zero-Mode Waveguide Enhanced Non-Fluorescent Quencher)
  misc_feature 14: note = linked to quencher molecule IABkFQ (Internal Abasic Site Cleaving Quencher)
tccacgggatagcc

SEQ ID NO: 29 - DNA, 20 bp, synthetic construct
  source 1..20: mol_type = other DNA
atcatgagttcacatgtccg

SEQ ID NO: 30 - DNA, 20 bp, synthetic construct
  source 1..20: mol_type = other DNA
cgtaggagtttggaccgtgt

SEQ ID NO: 31 - DNA, 26 bp, synthetic construct
  source 1..26: mol_type = other DNA
  misc_feature 1: note = labeled with FAM (6-carboxyluorescein, a fluorescent dye)
  misc_feature 9..10: note = linked to quencher molecule ZEN (Zero-Mode Waveguide Enhanced Non-Fluorescent Quencher)
  misc_feature 26: note = linked to quencher molecule IABkFQ (Internal Abasic Site Cleaving Quencher)
ctgagaggaaggtcccccacattgga

SEQ ID NO: 32 - DNA, 22 bp, synthetic construct
  source 1..22: mol_type = other DNA
aatattggtcaatgggcgagag

SEQ ID NO: 33 - DNA, 24 bp, synthetic construct
  source 1..24: mol_type = other DNA
cacgtagtgtccgttattccctta

SEQ ID NO: 34 - DNA, 17 bp, synthetic construct
  source 1..17: mol_type = other DNA
  misc_feature 1: note = labeled with FAM (6-carboxyluorescein, a fluorescent dye)
  misc_feature 9..10: note = linked to quencher molecule ZEN (Zero-Mode Waveguide Enhanced Non-Fluorescent Quencher)
  misc_feature 17: note = linked to quencher molecule IABkFQ (Internal Abasic Site Cleaving Quencher)
tccttcacgctacttgg

(19) United States
(12) Patent Application Publication
(10) Pub. No.: US 2025/0333801 A1
(43) Pub. Date: Oct. 30, 2025

(54) ASSAYS, KITS AND METHODS FOR DETECTION OF CONTAMINATION

(71) Applicant: PURDUE RESEARCH FOUNDATION, West Lafayette, IN (US)

(72) Inventors: Mohit Verma, West Lafayette, IN (US); Jiangshan Wang, West Lafayette, IN (US); Mohsen Ranjbaran, San Diego, CA (US)

(21) Appl. No.: 18/866,530

(22) PCT Filed: May 17, 2023
(86) PCT No.: PCT/US2023/067141

§ 371 Date: Nov. 16, 2024

Related U.S. Application Data
(60) Provisional application No. 63/342,906, filed on May 17, 2022.
(60) Provisional application No. 63/441,932, filed on Jan. 30, 2023.

(51) Int. Cl. C12Q 1/689

(52) U.S. Cl. CPC C12Q 1/689

(57) ABSTRACT

Loop-mediated isothermal amplification assays, kits, and methods that target and/or detect the presence of a fecal indicator bacteria in a sample. These assays, kits, and methods can be portable and capable of providing fast (within 60 minutes) results in the field, eliminating the need for a laboratory and other complex equipment.

### PRIORITY

This application is related to and claims the priority benefit of (1) U.S. Provisional Patent Application No. 63/342,906 filed May 17, 2022, and (2) U.S. Provisional Patent Application No. 63/441,932 filed Jan. 30, 2023. The contents of the aforementioned applications are hereby incorporated by reference in their entireties into this disclosure.

### STATEMENT OF GOVERNMENT SUPPORT

This invention was made with government support under USDA-AMS-TM-SCBGP-G-20-0003 awarded by the United States Department of Agriculture (USDA). The government has certain rights in the invention.

### TECHNICAL FIELD

The present disclosure includes loop-mediated isothermal amplification (LAMP) assays comprising a primer set that targets a deoxyribonucleic acid fragment of fecal indicator bacteria (FIB) in a sample and allows for single-step identification of the presence or absence of the FIB in the sample, which is indicative of the presence or absence of fecal contamination. Kits comprising the LAMP assay are also provided, as are methods of monitoring fecal contamination and methods for microbial source tracking.

### SEQUENCE LISTINGS

The sequences herein are also provided in computer readable form encoded in a file tiled herewith and incorporated herein by reference. The information recorded in computer readable form is identical to the written Sequence Listings provided below, pursuant to 37 C.F.R. § 1.821(f).

### BACKGROUND

This section introduces aspects that may help facilitate a better understanding of the disclosure. Accordingly, these statements are to be read in this light and are not to be understood as admissions about what is or is not prior art.

Fecal contamination of fresh produce from animal sources is a public health concern due to the risk of foodborne illnesses and foodborne outbreaks caused by fecal contamination of fresh produce represent a serious concern to public health and the economy. For the past few decades, the incidence of food-borne illness associated with fresh produce has increased and foodbome pathogens have been associated with a significant number of multistate outbreaks in the United States. Fresh produce is typically cultivated in open fields, making it susceptible to environmental reservoirs of foodbome pathogens during production (such as poorly composted animal manures, subpar irrigation water, encroachment of wild animals, and bioaerosols from nearby animal operations).

The majority of foodbome pathogens linked to fresh produce (i e., diarrheagenic Escherichia coli, Salmonella enterica, and Listeria monocytogenes) are enteric in origin and fecal contamination can occur anywhere along the farm-to-fork chain. Alegbeleye et al., Sources and contamination routes of microbial pathogens to fresh produce during field cultivation: a review, Food Microbiology 73: 177-208 (2018); Bozkurt et al., Assessment of microbial risk during Australian industrial practices for Escherichia coli O157:H7 in fresh cut-cos lettuce: a stochastic quantitative approach, Food Microbiology 95: 103691 (2021); Hoelzer et al., Emerging needs and opportunities in foodbome disease detection and prevention: from tools to people, Food Microbiology 75: 65-71 (2018); Qi et al., Glove-mediated transfer of Listeria monocytogenes on fresh-cut cantaloupe, Food Microbiology 88: 103396 (2020). The use of poorly composted animal manures, substandard irrigation waters, wild animal encroachment, and the spread of airborne bacteria (bioaerosols) from nearby livestock operations are all potential points of entry while growing fresh produce. Alegbeleye et al. (2018), supra; Chen et al., Prevalence and methodologies for detection, characterization and subtyping of Listeria monocytogenes and L. ivanovii in foods and environmental sources, Food Science & Human Wellness 6: 97-120 (2017); Li et al., Filtration assisted pretreatment for rapid enrichment and accurate detection of Salmonella in vegetables, Food Science & Human Wellness 12: 1167-1173 (2023).

As the consumption of fresh produce increases, public health officials and organizations have pushed for improvements in food safety procedures and environmental assessments to reduce the risk of contamination. Visual inspects and the establishment of “buffer zones” between animal feeding operations and producing fields are the current best practices for environmental assessments. However, a generalized distance guideline and visual inspects may not be enough to account for all environmental risk variables.

Many fresh produce organizations have devised and implemented safety practices and protocols to reduce potential sources of contamination. The California Leafy Greens Marketing Agreement (LGMA) Food Safety Standards, for example, specify that the best practice for environmental assessments is to inspect the production field and surrounding area for potential animal hazards or other sources of human pathogens of concern. A “buffer zone” of 400 feet for animal feeding operations (less than 1,000 animals) or 1200 feet for concentrated animal feeding operations (1,000-80,000 animals) around the production field is required to prevent pathogen transmission from animals to crops (LGMA, 2021). However, the fact that each farm has a unique combination of environmental risk variables (e.g., topography, land-use interactions, and weather) makes this generalized distance guideline difficult to justify. Strawn et al., Landscape and Meteorological Factors Affecting Prevalence of Three Food-Borne Pathogens in Fruit and Vegetable Farms, Applied Environmental Microbiology 79: 588-600 (2013a); Strawn et al., Risk Factors Associated with Salmonella and Listeria monocytogenes Contamination of Produce Fields, Applied Environmental Microbiology 79: 7618-7627 (2013b). Furthermore, while these practices were initially used to limit food safety risks for preharvest production, applying them to all fields without specificity would elevate production costs (for low-risk fields) and raise produce safety concerns (for high-risk fields). Indeed, LGMA acknowledges that there is limited information on which to base this recommendation, and ideally an appropriate “buffer zone” should be customized to each farm. Hoar, Developing buffer zone distances between sheep grazing operations and vegetable crops to maximize food safety, Center for Produce Safety (2011); Strawn et al. (2013b), supra.

In the majority of cases, the concentration of the enteric pathogens is relatively low, which makes it difficult to identify and enumerate their presence. Lemarchand & Lebaron, Occurrence of Salmonella spp and Cryptosporidium spp in a French coastal watershed: relationship with fecal indicators, FEMS Microbiology Letters 218(1): 203-209 (2003); Ferone et al., Microbial detection and identification methods: bench top assays to omics approaches. Comprehensive Review Food Science & Food Safety 19(6): 3106-3129 (2020). Additionally, due to the high level of heterogeneity in fresh produce products, pretreatments are typically required where the pretreatment process also dilutes the pathogens. U.S. Food & Drug Administration (FDA), Bacteriological Analytical Manual (BAM), FDA (2021); Ferone et al. (2020), supra. Enteric pathogens can enter a viable but non-culturable state (VBNC) and maintain a low level of metabolic activity without growing on typical microbial media, therefore escaping detection using culture-based approaches Martinez-Vaz et al., Enteric pathogen-plant interactions: molecular connections leading to colonization and growth and implications for food safety. Microbiology Environments 29(2): 123-135 (2014); Oliver, The viable but nonculturable state in bacteria, J Microbiology (Seoul, Korea), 43 Spec No 93-100 (2005).

Nevertheless, the presence of pathogenic enteric microorganisms on fresh produce poses a significant health risk to humans. Since it is difficult to quantify the abundance of all potential pathogens, it is common practice to only quantify the presence/absence of one or a few fecal indicator bacteria (FIB) (such as Escherichia coli, Enterococcus faecalis, and Bacteroidales) which are microorganisms that have been selected as indicators of fecal contamination. Brauwere & Servais, Modeling fecal indicator bacteria concentrations in natural surface waters: a review, Critical Reviews in Environmental Science & Technology 44(21): 2380-2453 (2014); Denis et al., Prevalence and trends of bacterial contamination in fresh fruits and vegetables sold at retail in Canada, Food Control 67: 225-234 (2016); Drozd et al., Evaluating the Occurrence of Host-Specific Bacteroidales, General Fecal Indicators, and Bacterial Pathogens in a Mixed-Use Watershed, J Environmental Quality 42: 713-725 (2013); Harris et al., Fecal Contamination on Produce from Wholesale and Retail Food Markets in Dhaka, Bangladesh, Am J Tropical Medicine & Hygiene 98: 287-294 (2017); Ordaz et al., Persistence of Bacteroidales and other fecal indicator bacteria on inanimated materials, melon and tomato at various storage conditions, Intemational J Food Microbiology 299: 33-38 (2019).

Conventional laboratory procedures for FIB detection include culture-based methods and DNA-based approaches and usually require an enrichment step that takes several hours. Hoadley & Cheng, The recovery of indicator bacteria on selective media. J Applied Bacteriology 37(1): 45-57 (1974); Li et al., Formation and Control of the Viable but Non-culturable State of Foodborne Pathogen Escherichia coli 0157, H7, Frontiers in Microbiology 11 (2020); Zhao et al., Current perspectives on viable but non-culturable state in foodbome pathogens, Frontiers in Microbiology 8 (2017). Culture-dependent approaches require the use of a microbiology lab and have limitations in detecting the VBNC state. Another limitation of the culture-based FIB approach is required ovemight incubation, which can significantly delay findings and prevent early warnings and prompt implementation of contamination control or mitigation steps. To quickly determine microbial contamination, molecular techniques such as PCR have been used. PCR-based approaches for monitoring FIB depend heavily on access to a laboratory, professional staff, and expensive equipment and, thus, are not conducive to rapid in-field assessment of contamination. Further, due to the low quantity of pathogen typically present, PCR techniques are likely to give a false negative result.

FIB, such as Escherichia coli, Enterococcus faecalis, and Bacteroidales, are commonly used to assess microbial water quality. Allende et al., Quantitative microbial exposure modelling as a tool to evaluate the impact of contamination level of surface irrigation water and seasonality on fecal hygiene indicator E. coli in leafy green production, Food Microbiology 75: 82-89 (2018); Kundu et al., Quantitative microbial risk assessment to estimate the risk of diarrheal diseases from fresh produce consumption in India, Food Microbiology 75: 95-102 (2018); Topalcengiz & Danyluk, Assessment of contamination risk from fecal matter presence on fruit and mulch in the tomato fields based on generic Escherichia coli population, Food Microbiology 103: 1039562022 (2022); Truchado et al., Suitability of different Escherichia coli enumeration techniques to assess the microbial quality of different irrigation water sources, Food Microbiology 58: 29-35 (2016). Bacteroidales are a common target as they are confined to warm-blooded animals and are a major component of gut microflora. Bernhard & Field, A PCR assay to discriminate human and ruminant feces on the basis of host differences in bacteroides-prevotella genes encoding 16S rRNA. Applied Environmental Microbiology 66(10): 4571-4574 (2000). Furthermore, as obligate anaerobes, Bacteroidales are unable to proliferate in standard atmospheric conditions.

Molecular techniques such as PCR and quantitative PCR (qPCR) are currently applied to detect Bacteroidales. The PCR-based assays target either highly conserved regions of the 16S gene or variable regions representing individual hosts. Bacteroidales assays have been extensively used as general indicators of microbiological water quality. These methods are advantageous because of their high levels of precision, specificity, and sensitivity. Recently, a few studies have also attempted to use Bacteroidales as a target to detect possible fecal contamination in fresh produce.

Compared to PCR, loop-mediated isothermal amplification (LAMP) enables simpler detection of microorganisms in environmental samples. Notomi et al., Loop-mediated isothermal amplification of DNA, Nucleic Acids Research 28(12): e63 (2000). Due to the inherent characteristic of LAMP Bst DNA polymerase, only a single temperature (in the range of 60-65° C.) is required for the reaction to be conducted (as opposed to cycling of temperature, which is required for PCR). The reaction could be carried out in the field using a cost-effective, simple heat source, such as an incubator or a water bath, in contrast to the expensive thermocyclers needed by traditional PCR methods. Furthermore, the Bst polymerase is resistant to common PCR inhibitors found in unpurified environment samples, enabling direct measurements. As a result, LAMP has been widely used as a point-of-care assay for applications in food safety and diagnostics of human and animal health. Incorporating a colorimetric dye (e.g., EBT, phenol red) in LAMP assays enables color changes that are visible to the naked eye.

A human-associated Bacteroides detection device based on fluorescent-LAMP for monitoring human fecal contamination in water has been developed. However, this approach requires a relatively long assay time (80 minutes) and a transilluminator to visualize the fluorescence.

While LAMP does show promise as an effective diagnostic tool, a major limitation of using LAMP as a mainstream assay for pathogen screening is the occurrence of false positives—either due to poor reagent handling or carryover contamination from previous experiments. Additionally, the accuracy of LAMP is heavily dependent on the primers used and, prior to this disclosure, optimal primer sets had yet to be identified. Indeed, designing LAMP primers has proven challenging. Accordingly, there remains a need to provide a cost-effective, rapid, and accurate in-situ assay to detect the presence of Bacteroidales and assess the risk of fecal contamination in fresh produce. Furthermore, there is a need for a rapid and easy to deploy method of assessing a risk of and/or monitoring fecal contamination in fresh product production.

### SUMMARY

Loop-mediated isothermal amplification (LAMP) assays are provided. A LAMP assay can comprise at least one LAMP primer set that targets a deoxyribonucleic acid (DNA) fragment of fecal indicator bacteria (FIB) in a sample. The assay can allow for single-step identification of the presence or absence of the FIB in the sample. The presence of FIB can indicative of the presence of a foodborne pathogen in the sample, and the absence of FIB can be indicative of the absence of a foodbome pathogen in the sample.

The FIB can be Bacteroidales, Escherichia coli, and/or Enterococcus faecalis. The FIB can be Bacteroidales.

The at least one primer set can comprise one or more primers of SEQ ID NO: 4 and SEQ ID NO: 5. The at least one primer set can comprise one or more primers of SEQ ID NO: 6 and SEQ ID NO: 7. The at least one primer set can comprise one or more primers of SEQ ID NO: 8 and SEQ ID NO: 9. The at least one primer set can comprise primers of SEQ ID NOS: 4-9.

In certain embodiments, the assay can process and provide a visual result in 60 minutes or less. The visual result can be indicative of the presence or absence of the FIB in the sample. The visual result can be a color-coded or colorimetric result. The at least one LAMP primer set can be coupled with a colorimetric reagent. The colorimetric reagent can be phenol red.

In certain embodiments, the LAMP assay further comprises a fluorescent indicator. The targeted DNA fragment can comprise a species-specific gene (such as, for example, a 16S rRNA gene sequence). The targeted DNA fragment of FIB can comprise a 16S rRNA gene sequence.

Each of the LAMP primer sets can have a limit of detection (LoD) of at least about 20 copies/cm² surface area of a collection surface from which the sample was obtained. Each of the LAMP primer sets can have a LoD of at least about 17 copies/cm² surface area of a collection surface from which the sample was obtained. Each of the LAMP primer sets can have a LoD of at least about 10³-10⁴ copies/cm² surface area of a collection surface from which the sample was obtained.

Kits comprising the LAMP assays hereof are also provided. A kit can comprise at least one LAMP primer set (e.g., any of the primer sets described herein); at least one swab for obtaining the sample; and a heating element to initiate amplification of the targeted DNA fragment when the at least one LAMP primer set and the sample are combined. The heating element can be a water bath. The kit can comprise one or more containers with a reaction mixture therein (e.g., a master mix therein). In certain embodiments, a container can be a sealable container. The one or more containers can each comprise a vial, a microcentrifuge tube, or a tube strip.

The kit can further comprise a fluorescent indicator; and a fluorescent reader, an ultraviolet light reader, or a camera to provide colorimetric result data indicative of the presence or absence of FIB in the sample. The at least one LAMP primer set can be coupled with a colorimetric reagent. The colorimetric reagent can be, for example, phenol red.

The kit can be portable and capable of use in a non-laboratory setting. In certain embodiments, the kit further comprises a plurality of collection flags for the collection of bioaerosol samples. Each collection flag can comprise a film affixed to a support at a distance away from an end thereof such that, in use, the support can anchor the film a distance above a surface of an area in which the support is positioned.

In certain embodiments, the kit can further comprise a control or reference for comparison with reacted samples. The control or reference can determine a baseline against which the visual results of the samples can be compared and/or measured. For example, the control can be a container with master mix therein, but no LAMP assay. In certain embodiments, the reference is a reference card showing color images of reacted and unreacted assays so that a user can compare reacted samples with the colors shown in the reference images to determine if a reaction occurred. A LoD of the LAMP primer set can be about 17 copies of FIB per cm² of surface area of the film.

Methods of monitoring fecal contamination are also provided. In certain embodiments, a method of monitoring fecal contamination comprises: providing at least one LAMP primer set hereof; obtaining a sample from a target; combining the sample and the at least one LAMP primer set into a mixture; heating the combination to initiate amplification of the targeted DNA fragment; and detecting a visual result in the heated combination indicative of the presence or absence of the targeted FIB in the sample; wherein detection of a visual result indicative of the presence of the targeted FIB in the sample is also indicative of the presence of a foodbome pathogen in the sample, and the absence of FIB is indicative of the absence of a foodbome pathogen in the sample. The FIB can be Bacteroidales and the at least one LAMP primer set can comprise primers of SEQ ID NOS. 4-9.

The target can comprise a field and the sample can comprise a plurality of samples collected from various locations across the field. The target can comprise a planted field prior to harvest. The target can comprise an unplanted field prior to growing season. In certain embodiments, if the presence of the targeted FIB is detected in the sample, the method can further comprise destroying a crop planted in the field; or if the absence of the targeted FIB is detected in the sample, the method can further comprise harvesting the crop planted in the field. In certain embodiments, if the presence of the targeted FIB is detected in the sample, the method can further comprise planting crops in the field that are not for human raw consumption. In certain embodiments, if the presence of the targeted FIB is detected in the sample, the method can further comprise performing the microbial source tracking method. In certain embodiments, if the presence of the targeted FIB is detected in the sample, the method can further comprise treating the field to remediate any fecal contamination. If the absence of the targeted FIB is detected in the sample, the method can further comprise planting a crop in the field.

In certain embodiments, the method further comprises identifying the target (i.e., a fresh produce crop or a field) as “high-risk” if the visual result equates with a surface concentration of the target FIB at or about 4 orders of magnitude greater than a “low-risk” value. The “low-risk” value can be a control value. The “low-risk” value can be at or about 2 copies/cm² of surface area. The “low-risk” value can be less than 17 copies/cm² of surface area of a collection surface from which the sample was obtained (e.g., 16 copies/cm², 15 copies/cm², 15 copies/cm², 14 copies/cm², 13 copies/cm², 12 copies/cm², 11 copies/cm², 10 copies/cm², 9 copies/cm², 8 copies/cm², 7 copies/cm², 6 copies/cm², 5 copies/cm², 4 copies/cm², 3 copies/cm², 2 copies/cm², 1 copies/cm², or less than 1 copies/cm²).

Methods of microbial source tracking are also provided. In certain embodiments, the method of microbial source tracking comprises: providing a first LAMP primer set that targets a DNA fragment of a first targeted FIB in a sample; obtaining a sample from a target; combining the sample and first LAMP primer set into a mixture; heating the combination to initiate amplification of the targeted DNA fragment; and detecting a visual result in the heated combination indicative of the presence or absence of the first targeted FIB in the sample, wherein the first targeted FIB is an FIB of a first species and the first LAMP primer set is species-specific to the first species. The first LAMP primer set can be coupled with a colorimetric reagent of a first color such that a visual result can be indicative of the presence of the first targeted FIB in the sample comprises the first color. The LoD of the assay in providing a result indicative of the presence of the targeted FIB can be as low as about 17 copies/cm² surface area.

The method of microbial source tracking can further comprise providing a second LAMP primer set that targets a DNA fragment of a second targeted FIB in a sample; combining the sample and the second LAMP primer set into a mixture; heating the combination to initiate amplification of the targeted DNA fragment; and detecting a visual result in the heated combination indicative of the presence or absence of the second FIB in the sample, wherein the second targeted FIB is an FIB of a second species and the second LAMP primer set is species-specific to the second species. The second LAMP primer set can be coupled with a colorimetric reagent of a second color such that a visual result indicative of the presence of the second targeted FIB in the sample comprises the second color.

The visual result can be provided in about 60 minutes or less (such as in 60 minutes or less) of initiating the heating step. The sample can be a bioaerosol sample.

In certain embodiments, the target comprises a field and the method further comprises: collecting one or more collection flags from the field, wherein each collection flag comprises a film affixed to a support; and swabbing the sample of a surface of the film of each collection flag. The film of a collection flag can be a transparent film. The film can comprise a plastic.

Each collection flag can be encoded with a unique identifier that is indicative of a location in the field in which the collection flag was positioned. In certain embodiments, the method further comprises generating a map of the visual results by associating each visual result with the unique identifier of the collection flag from which the respective sample was obtained.

Detecting a visual result can further comprise analyzing colorimetric data in the visual result using one or more of a fluorescent reader, an ultraviolet light reader, or a camera. In certain embodiments, the method further comprises tracking sources of contamination by using primer sets comprising host-associated 16S rRNA gene sequences.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, figures, and tables.

FIGS. 1A-1E show data related to the characterization of LAMP primer sets, with FIG. 1A showing fluorometric result from LAMP primer set Universal. Bacteroidales.16s rRNA.1 (“Primer Set 1”) using genomic DNA extract from pure culture of Bacteroides fagilis, FIG. 1B showing fluorometric performance of Primer Set 1 using stool extractions, FIG. 1C showing colorimetric result from Primer Set 1 using genomic DNA extract from pure culture, FIG. 1D showing fluorometric results for primer set Universal. Bacteroidales.16s rRNA.2; and FIG. 1E showing fluorometric results for primer set Universal. Bacteroidales.16s rRNA.3.

FIG. 2 shows graphical data related to limit of detection (LoD) characterization of LAMP (Primer Set 1) assay.

FIG. 3 shows LoD characterization of qPCR (GenBac3) assay.

FIG. 4 shows a LoD characterization of LAMP (Primer Set 1) colorimetric assay. The colors identified to the right of each row apply to the entire row, except where specifically indicated otherwise (e.g., the yellow well indicated in the 25 copies/reaction row).

FIG. 5 illustrates the fabrication process of collection flags, with subpart A showing the starting materials; subpart B showing the cutting of transparent film to 5 cm×30 cm strips; subpart C showing four pieces of film being stapled together at the edge to form a loop; subpart D showing a bamboo skewer (support) being inserted through the loop to make a collection flag; and subpart E showing a completed collection flag ready for deployment.

FIG. 6 shows satellite images of the field study, with subpart A showing the cattle unit, subpart B showing the swine unit, subpart C showing the poultry unit, and subpart D showing the Tt value of each LAMP reaction converted to log₁₀ (copies/cm²) via a linear fit to log-transformed concentrations.

FIGS. 7A-7D show fluorometric LAMP (Primer Set 1) assays using lettuce leaves swab resuspension solution.

FIGS. 8A-8D show fluorometric LAMP (Primer Set 1) assays using collection flag swab resuspension solution.

FIGS. 9A-9D show qPCR (GenBac3) using lettuce leaves swab resuspension solution.

FIGS. 10A-10D show qPCR (GenBac3) using collection flags swab resuspension solution.

FIG. 11 shows on-site colorimetric LAMP (Primer Set 1) assay comparison to lab LAMP and qPCR.

FIG. 12 shows images of LAMP assay deployed on-site, with the swabbing of collection flags (subparts A and B), adding swab resuspension into the reaction mix (subparts C and D), and running the LAMP assay with an Anova Culinary Precision Cooker on site (subparts E and F).

FIGS. 13A-13C show fecal contamination mapping using qPCR (May 2021), where the Ct value of each qPCR reaction was converted to log₁₀ (copies/cm²) via a linear fit to log-transformed concentrations.

FIGS. 14A-14C show fecal contamination mapping using qPCR (August 2021), where the Ct value of each qPCR reaction was converted to log₁₀ (copies/cm²) via a linear fit to log-transformed concentrations.

FIG. 15 shows a qPCR calibration curve, where the Ct values were calculated using software qPCRsoft 4.1 (baseline correction: 5, auto threshold) (Analytik Jena, Germany).

FIG. 16 shows a scatter plot of microbial source tracking results.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, tables, and figures and specific language will be used to describe the same. It will nevertheless be understood that no limitation of the scope of this disclosure is thereby intended.

The present disclosure includes various assays, kits, and methods to target and/or detect and/or treat the presence or absence of Bacteroidales, such as to assess fresh produce fecal contamination. As used herein, the term “fresh produce” includes both cut and whole fresh fungi, fruits, and vegetables including, for example and without limitation, greens, celery, berries, and the like. The term “fresh” means that the food is in its raw state and has not been frozen or subjected to any form of thermal processing or any other form of preservation (other than potentially post-harvest pesticides, the application of a mild chlorine wash or mild acid wash, or treatment with ionizing radiation).

These assays (and methods of assessing risk of fecal contamination using such assays) can be portable, disposable, and capable of providing fast and accurate results in the field without the need for a laboratory and other complex equipment.

Additionally the assays presented herein provide rapid and accurate results (as compared to conventionally available assays and other methodologies). Perhaps more specifically, the novel primer sets of the assays, kits, and methods hereof can decrease testing time to less than 60 minutes, thus providing fast and accurate results. In certain embodiments, the present assays can detect bioaerosols present in samples at levels of below 10,000 copies/cm². In certain embodiments, the LoD can be as low as about 17 copies/cm².

In at least one embodiment, a portable assay or method using the same comprises a loop-mediated isothermal amplification (LAMP) assay that utilizes novel primers (e.g. primer sets) for detecting and/or quantifying fecal indicator bacterial (FIB) present within a sample. Data establishing baseline thresholds of FIB contamination as it correlates to the presence or absence of foodborne pathogens within a test group (e.g., a crop or pre-planted field) are also provided, which, for example, can be considered in connection with results from the novel LAMP assays hereof to determine if fecal contamination is present within a crop or a pre-planted field.

Also disclosed herein are detection methods using LAMP assays that specifically target and detect the presence of a FIB (e.g., Bacteroidales) from produce-collected or flag-collected samples to provide a risk assessment of fecal contamination. Accordingly, the assays, kits and methods hereof can be used to rapidly and accurately diagnose fecal contamination in a test group (such as a fresh produce crop in a field) such that mitigating steps can, where desired, be taken. Additionally, the assays, kits and methods hereof can be used to monitor fecal contamination in fresh produce production and for microbial source tracking.

### LAMP Assays and Kits

In at least one embodiment, a portable assay or method using the same comprises a LAMP assay that utilizes novel primers (e.g., primer sets). Also disclosed herein are detection methods using LAMP assays that can specifically target and detect the presence of FIBs such as Bacteroidales, Escherichia coli, and/or Enterococcus faecalis from samples taken from a field (whether via a leaf or produce swab, or from a collection flag as described herein). The assays, kits and methods hereof can be used to rapidly and accurately identify in a non-laboratory setting if fecal contamination is present in a field (i.e., if a field is “high risk” for fecal contamination).

“High-risk” as used herein means a target that measures as having a high concentration of FIB (e.g., at or about 4 orders of magnitude higher than a “low-risk” threshold) and, thus, is contaminated with fecal matter and foodbome pathogens. “Low-risk” as used herein means a target that measures as having a low concentration of FIB and, thus, is not likely contaminated with feces and/or foodborne pathogens to the extent fresh produce grown therein would result in consumer illness. In certain embodiments, a “low-risk” threshold is the targeted FIB being present in at or less than 2 copies/cm² of surface area on the collection surface. In certain embodiments, a “low-risk” threshold is the targeted FIB being present in at or less than 10 copies/cm² of surface area on the collection surface. In certain embodiments, the “low-risk” threshold is the FIB being present in less than 17 copies/cm² of surface area on the collection surface (e.g., 16 copies/cm², 15 copies/cm², 15 copies/cm², 14 copies/cm², 13 copies/cm², 12 copies/cm², 11 copies/cm², 10 copies/cm², 9 copies/cm², 8 copies/cm², 7 copies/cm², 6 copies/cm², 5 copies/cm², 4 copies/cm², 3 copies/cm², 2 copies/cm², 1 copies/cm², or less than 1 copies/cm²).

LAMP uses 4-6 primers that can recognize 6-8 distinct regions of target deoxyribonucleic acid (DNA) for a highly specific amplification reaction. A strand-displacing DNA polymerase initiates synthesis and two specifically designed primers form “loop” structures to facilitate subsequent rounds of amplification through extension on the loops and additional annealing of primers. DNA products are typically long (>20 kb) and formed from numerous repeats of the short (80-250 bp) target sequence, connected with single-stranded loop regions in long concatamers. These products are not typically appropriate for downstream manipulation, but the achievable target amplification can be so extensive that numerous modes of detection are possible.

Real-time fluorescence detection using intercalators or probes, lateral flow, and agarose gel detection, for example, are all directly compatible with LAMP reactions. Instrumentation for LAMP typically requires consistent heating to the desired reaction temperature and, where desired, real-time fluorescence for quantitative measurements. Optimized settings for running LAMP assays on isothermal instruments are known in the art, and the assay can be performed using the techniques described in detail in at least Notomi, T. et al., “Loop-mediated isothermal amplification of DNA,” Nucleic Acids Res. 2000, Jun. 15; 28(12): e63 (doi: 10.1093/nar/28.12.e63) and Nagamine, K et al., “Accelerated reaction by loop-mediated isothermal amplification using loop primers,” Mol. Cell. Probes 2002; 16: 223-229, both of which are incorporated herein by reference in their entireties.

In certain instances, LAMP can be so prolific that the products and byproducts of these reactions can be visualized by the naked eye. For example, magnesium pyrophosphate produced during the reaction can be observed as a white precipitate or added indicators (e.g., calcein or hydroxynaphthol blue) can be used to signal a positive reaction or an indicative pH change. In certain embodiments, the visual result can be provided in 60 minutes or less and is indicative of the presence or absence of the targeted FIB in the sample.

In certain embodiments, the visual result is indicative of the presence of the targeted FIB in the sample where the concentration of the targeted FIB in the sample (i.e., that collected from a collection surface area (e.g., a leaf or a collection flag surface from which the sample is collected)) is greater than the LoD of the assay. In certain embodiments, the LoD of the assay is about 17 copies of FIB per cm² (such as 17 copies/cm²) of a collection surface area from which the sample was obtained. In certain embodiments, the LoD of the assay is about 20 copies/cm² (such as 20 copies/cm²) of a collection surface area from which the sample was obtained. In certain embodiments, the LoD of the assay is about 100 copies/cm² (such as 100 copies/cm²) of a collection surface area from which the sample was obtained. In certain embodiments, the LoD of the assay is about 1250 copies/cm² (such as 1250 copies/cm²) of a collection surface area from which the sample was obtained. In certain embodiments, the LoD of the assay is about 10³ copies/cm² (such as 10³ copies/cm²) of a collection surface area from which the sample was obtained. In certain embodiments, the LoD of the assay is about 10³-10⁴ copies/cm² (such as 10³-10⁴ copies/cm²) of a collection surface area from which the sample was obtained.

The visual result can be color-coded or colorimetric. In certain embodiments, the LAMP assay can be coupled with a colorimetric reagent that is sensitive to magnesium or pH and allows for visualization of the result with the naked eye and/or quantification using a camera. Such colorimetric reagents, for example, can include a phenol red. In certain embodiments, the LAMP assays hereof are coupled with a colorimetric reagent. In certain embodiments, the LAMP assays hereof are coupled with a colorimetric reagent that has a limit of detection (LoD) of 1250 copies of DNA per reaction. In an exemplary embodiment, the primers described herein are coupled with a composition comprising phenol red, such as, for example and without limitation, Warmstart® LAMP 2×Master Mix.

Warmstart® LAMP 2×Master Mix, which contains phenol red, is characterized by its transition from pink to yellow as the LAMP reaction occurs and the pH decreases.

As noted above, conventional precedent does not allow for the presence of foodbome pathogens and/or fecal pollution to be accurately detected or monitored in a field setting. The LAMP assays for FIB provided herein, however, are accurate and sensitive.

LAMP assays hereof offer at least six advantages: (1) they can be conducted on the farm/in the field using a simple consumer-grade water bath; (2) they can provide a visual readout and, thus, allow for analysis with the naked eye (e.g., in some instances a quick and simple visual “yes/no” result readout); (3) they provide a response in at or less than about 60 minutes; (4) they do not require sample processing (e.g., extraction or purification of nucleic acids); (5) they allow for detection of FIB present in a sample and, in particular Bacteroidales, with the naked eye with as few as about 17 copies of Bacteroidales per cm² of the surface in the field; and (6) they utilize non-pathogenic FIBs for indicators.

In certain embodiments, the LAMP assay comprises at least one LAMP primer set that targets a DNA fragment of a FIB. The FIB can be any FIB. In certain embodiments, the FIB is Bacteroidales, Escherichia coli, and/or Enterococcus faecalis. In certain embodiments, the FIB is Bacteroidales. The DNA fragment of FIB can comprise a 16S rRNA gene sequence.

Certain features of Bacteroidales make them superior to other FIB including high prevalence in feces (constituting 30%-40% of total fecal bacteria, 10⁹ to 10¹¹ colony forming units (CFU)/g), obligate anaerobicity (preventing their growth and multiplication in the ambient environment), low natural abundance from non-fecal sources, and high host-specificity (various sequences of the 16S rRNA gene have been designed to detect fecal pollution from specific hosts). Mascorro et al., Bacteroidales as Indicators and Source Trackers of Fecal Contamination in Tomatoes and Strawberries, J Food Protection 81: 1439-1444 (2018); Ordaz et al. (2019), supra. Bacteroidales outnumber facultative anaerobes, such as Escherichia coli and Entercoccus faecalis (two other commonly used FIBs) by a factor of 10³-10⁴ and 10⁴-10⁵, respectively. Gorbach, Microbiology of the Gastrointestinal Tract. In: Baron, S. (Ed.), Medical Microbiology (4th ed.), University of Texas Medical Branch at Galveston (1996). As such, Bacteroidales detection can provide at least 1000 times better sensitivity than detection using other common FIB when a technique with the same or an equivalent LoD is used. Additionally, it is possible to directly detect Bacteroidales (e.g., to evaluate fecal contamination in a sample) without prolonged cultivation, it is non-pathogenic, and it provides the ability to assess the presence of multiple pathogens at once.

The targeted DNA of the FIB is, preferably, a DNA segment or region that has little to no homology with non-targeted bacteria. While some such gene targets are known, others such as those listed in Table 2 below were newly identified by the present investigators.

In certain embodiments, the DNA fragment of the FIB can be species-specific. Accordingly, the primer set of the assay can be configured to be species-specific as well, such that a visual result indicating the presence of the FIB also confirms the species from which such FIB originated. For example, Bacteroidales contain host-associated 16S rRNA gene sequences that have species-specific characteristics. Table 6 below lists several primer sets that are cattle-specific Bacteroidales (SEQ ID NOS: 23-25), swine-specific Bacteroidales (SEQ ID NOS: 26-28), human-specific Bacteroidales (SEQ ID NO: 29-31), and poultry-specific Bacteroidales (SEQ ID NOS: 32-34). Accordingly, the LAMP assay can comprise two or more of a first primer set comprising one or more primers of SEQ ID NOS: 23-25, a second primer set comprising one or more primers of SEQ ID NOS: 26-28, a third primer set comprising one or more primers of SEQ ID NOS: 29-31, and a fourth primer set comprising one or more primers of SEQ ID NOS: 32-34. Where each of the primer sets are coupled with a different colorimetric indicator, a positive result indicative of that particular primer set will not only indicate that Bacteroidales is present within the sample but will also indicate its species of origin. This could be particularly useful where a crop-site is located near several potential contamination sites (e.g., a swine farm to the East and a human sewage plant to the South) as it can provide valuable information regarding the source of contamination which can be leveraged to effectively remediate and/or prevent the same.

Each LAMP primer set of the assay is designed to target and amplify the targeted gDNA from a targeted FIB, while maintaining little to no amplification of other bacteria or negative samples. Each LAMP primer set can include 4 to 6 DNA primers (however the number of primers used can be modified, as desired). The at least one LAMP primer set can comprise 4 primers. The at least one LAMP primer set can comprise 6 primers. In each instance, the primer set is directed to the FIB of interest.

In certain embodiments, the LAMP primer set(s) each comprise a LAMP primer set listed in Table 2 or 6. In certain embodiments, the FIB is Bacteroidales and the primer set comprises one or more primers of SEQ ID NO: 4 and SEQ ID NO: 5. In certain embodiments, the FIB is Bacteroidales and the primer set comprises one or more primers of SEQ ID NO: 6 and SEQ ID NO: 7. In certain embodiments, the FIB is Bacteroidales and the primer set comprises one or more primers of SEQ ID NO: 8 and SEQ ID NO: 9. In certain embodiments, the FIB is Bacteroidales and the primer set comprises one or more primers of SEQ ID NOS: 4, 5, 6, 7, 8, and 9.

Any number of LAMP primer sets can be used in the same assay; for example, and without limitation, an assay can comprise a first LAMP primer set that targets a DNA fragment of an FIB from a first species, a second LAMP primer set that targets a DNA fragment of an FIB from a second species, and/or a third LAMP primer set that targets a DNA fragment of an FIB from a third species as noted above.

Additionally, the LAMP primer sets can comprise a combination of primer sets that each target DNA fragments of different FIBs. For example, a first primer set can target a DNA fragment of Bacteroidales, a second primer set can target a DNA fragment of Escherichia coli, and a third primer set can target a DNA fragment of Enterococcus faecalis.

The results of the LAMP assays hereof can, in some embodiments, be seen with the naked eye. While conventional versions of LAMP assays require SYBR Green staining for signal detection (which necessitates opening the tube after thermal incubation), the LAMP assays hereof can be performed with a turbidimeter (e.g., a Loopamp real-time turbidimeter) to detect a positive signal. A turbidimeter measures the relative clarity of the sample and does not require opening the tube, which reduces the risk of environmental diffusion and cross-contamination during gene amplification.

In certain embodiments, magnesium pyrophosphate produced during the reaction can be observed as a white precipitate or added indicators (e.g., calcein, magnesium-based indicators, or hydroxynaphthol blue) can be used to signal a positive reaction or an indication pH change.

In certain embodiments, the LAMP assays hereof can be coupled with or include indicators (e.g., colorimetric reagents or indicators) to allow for visual inspection of assay results without opening the reaction tube. Such assay results can provide a visual result that corresponds to the presence or absence of the targeted FIB in the sample. In some cases, the visual result is color-coded and/or colorimetric, and in other cases the result can be a letter, number, word, symbol, lines, or other representation indicative of the presence or absence of the targeted FIB. For example, if one of the LAMP primer sets is targeted to a DNA fragment unique to Bacteroidales, if Bacteroidales is present within the sample, the LAMP primer set will identify and amplify that DNA fragment. Where the assay further comprises an indicator associated with each LAMP primer set, the indicator associated with the Bacteroidales primer set will be easily detectable in the results.

Fluorescence can also be employed to facilitate signal detection. In at least one embodiment, the LAMP assays hereof further comprise fluorescent dye in the reagents mix for assay or a fluorescent tag coupled with the primers themselves. Fluorescent data/intensities can thereafter be collected (using thermocyclers or a fluorometer, for example) and analyzed. In the above non-limiting example where a loop primer is directed to a unique DNA fragment associated with Bacteroidales, a particular fluorescent indicator can be coupled with such primer so that visualization of the fluorescence of that particular fluorescent indicator is indicative of the sample being positive for Bacteroidales.

As noted above, colorimetric reagents can be coupled with the primer set(s) of the LAMP assays described herein. In certain embodiments, the colorimetric agent is pH sensitive (e.g., phenol red). While specific embodiments and examples are provided herein, it will be appreciated that any colorimetric reagent sensitive to pH or magnesium can be employed.

Where multiple primer sets are used in the same assay, and each primer set is directed to a different FIB or species, indicators can be used to easily identify in the visual results indicating which FIB is present in the sample and/or from which species the FIB originated. In at least one embodiment, for example, the first primer set can be labeled (at their 5′-ends, for example) with a stable, fluorescent material of a first intensity, the second primer set can be labeled with a stable, fluorescent material of a second intensity, and the third primer set can be labeled with a stable, fluorescent material of a third intensity using methods commonly known in the relevant arts. When the relevant primer set anneals to a complementary target amplicon (i.e., the DNA fragment of the targeted pathogen), the 5′-3′ exonucleolytic activity of DNA polymerase detaches the label from the primer, which results in an enhanced fluorescence signal at the intensity of the fluorescent material used for the primer set with which there was a match. Accordingly, assessment of the resulting intensity can identify which pathogen is present within the sample. While fluorescent indicators are described above, it will be appreciated that any type of indicators can be used with the novel assays of the present disclosure, including other indicators now known or hereinafter developed.

Additionally, certain embodiments of the LAMP assays can optionally utilize a fluorescent reader, an ultraviolet light reader, and/or a camera for signal detection and/or the display of assay results (e.g., where indicators are used). In these embodiments, the visual results may be color-metric and/or digitally provided, such as, for example, through a wireless device, laptop computer, or cell phone and may utilize WiFi, Bluetooth, or cellular data.

The LAMP assays (and primer sets thereof) can detect the targeted FIB DNA fragments in various sample types and, in certain embodiments, does not require that such samples be processed prior to running the assay. For example, a sample can comprise a simple water sample or an unprocessed sample obtained by swabbing a surface of fresh produce or a collection flag positioned (or previously positioned) within the target field. The ability to use unprocessed samples is advantageous for several reasons, at least one of which being that the assay translates easily to field use due to the ease of incubation. In certain embodiments, the samples, once collected, can be housed in a tube or vial containing a transport medium suitable for the collection, transport and/or handling of the specimen. For example, and without limitation, the transport medium can be liquid amies transport media.

Kits for testing one or more samples are also provided. Such kits can be configured for field use such as, for example, on-site at a growing operation, at a farm, or in a field. Accordingly, the kits can be portable and capable of use in a non-laboratory setting.

A kit can comprise at least one LAMP assay hereof and at least one swab (e.g., for obtaining a sample). Optionally, the kit can further comprise a heating element to initiate amplification of the targeted DNA fragment when the at least one LAMP primer set and the sample are combined. Additionally (or alternatively), the kit can comprise one or more containers, for example, for receiving the swab after collection of the sample and/or for providing an incubation environment where the reaction can occur.

The at least one LAMP assay can comprise one or more assays described herein. In certain embodiments, the LAMP assay comprises a LAMP primer set having one or more primers of SEQ ID NOS. 4-9. In certain embodiments, the primer set(s) of the LAMP assay comprises one or more primers of SEQ ID NOS: 4-9, SEQ ID NOS: 23-25, SEQ ID NOS: 26-28, SEQ ID NO: 29-31, and/or SEQ ID NOS: 32-34.

A swab of the kit can be any swab configured to obtain a sample from a plant or another collection means present in the targeted field. The swab can be a polyester-tipped swab. The swab can be a cotton-tipped swab. The swab can be any swab now known in hereinafter developed suitable for collecting the sample directly from a plant or from a collection means without introducing cross-contamination.

In certain embodiments, the container of the kit comprises a vial, a microcentrifuge tube, and/or tube strips. In at least one exemplary embodiment, the container can be used as the incubation environment for the collected sample and one or more LAMP primer sets (i.e., where the amplification reaction is performed on the collected sample). Accordingly, the container can contain a transport media or the like as is known in the art, and/or any additional reagents that are useful in facilitating the DNA amplification reaction and/or visualizing the results thereof. For example, in at least one embodiment, UDG/dUTP can be added to the media within the container to degrade leftover amplicons present therein after amplification of the targeted DNA. In certain embodiments, the container comprises a master mix. In certain embodiments, the container is pre-filled with a solution comprising (NH₄)₂SO₄ or Betaine, KCl MgSO₄, deoxynucloeside triphosphates (dNTPs), polysorbate 20 (e.g., Tween-20), a Bst 2.0 DNA polymerase, and/or a reverse transcriptase. In certain embodiments, the solution can further comprise phenol red. The solution can comprise 10 mM (NH₄)₂SO₄ or 25 mM Betaine, 50 mM KCl, 8 mM MgSO₄, 1.4 mM dNTPs, 0.1% v/v Tween-20, a BST 2.0 DNA polymerase (e.g., from New England Biolabs, Ipswich, MA) 8 U, 7.5 U RTx reverse transcriptase (e.g., from New England Biolabs, Ipswich, MA), and 100 mM phenol red.

In at least one embodiment, the container is sealable and is at least partially transparent such that visual results present within the container can be visualized without opening the container itself.

The assay of each kit can further comprise an indicator associated with each LAMP primer set. As described above, the LAMP primer sets can be configured to include the indicator (e.g., a fluorescent indicator coupled with an end of each primer) or the indicator can be added to the media housed by the container.

In certain embodiments, the indicator of each kit comprises a colorimetric reagent. For example, in certain embodiments, one or more of the LAMP primer sets can be coupled with a colorimetric reagent that is pH sensitive or magnesium sensitive. In certain embodiments, the colorimetric agent is phenol red.

The kit can further comprise a heating element to initiate amplification of the targeted DNA fragment when the at least one LAMP primer set and the sample are combined, for example, in the container. In certain embodiments, the heating element is a water bath. The kit can also, optionally, comprise a fluorescent reader, an ultraviolet light reader, or a camera to provide color metric result data indicative of the presence or absence of a targeted FIB in the sample.

The kit can further comprise a plurality of collection means for collecting samples. In certain embodiments, the collection means can comprise collection flags. A collection flag can comprise any device capable of being securely positioned in a targeted area for collecting bioaerosol samples. The collection flag can comprise a film or other material for receiving a sample, the film or other material affixed to a support configured to anchor the film a distance above a surface of atargeted area (e.g., a field being assessed). The film can be a transparent film. The film can comprise any material(s) and/or dimensions suitable for collecting samples provided the material is inert. In certain embodiments, the film comprises a smooth surface. In certain embodiments, the film comprises a 5 cm×30 cm strip that is wrapped around the support such that the film extends from the support about 15 cm in length. The film can be plastic. The film can comprise paper.

The support can be any material capable of being securely positioned in a targeted area (e.g., driven into the ground). In certain embodiments, the support is also capable of extending the film a distance above a surface of the targeted area (e.g., such that it is exposed to the air, but not necessarily touching the ground). The support can be made of wood, metal, plastic, or any other material sufficient to achieve this purpose, and can comprise any dimensions desired.

In terms of applicability, collection flags offer an advantage over direct produce sampling (e.g., leaf samples) by providing a standalone carrier for measuring fecal contamination. Environmental assessments are required at several phases throughout the production cycle, including before the vegetation planting. Growers must identify any potential sources of contamination in the production field and determine an adequate “buffer zone” to minimize environmental risks during production. The collecting and/or processing methods hereof that employ collection flags allow growers to conduct field assessments even with a product in the field. In certain embodiments, use of a collection flag allows for a LoD as low as about 17 copies of Bacteroidales per cm² of surface area of the film (copies/cm²). For reference, 25 grams of lettuce leaves would have approximately 1200 cm² around animal operations and is thus sufficiently sensitive for use in a commercial context.

### Methods

Methods of using the LAMP assays hereof are also provided. FIBs can be used as biomarker(s) for assessing fecal contamination levels of fresh produce and/or a field. Bacteroidales in particular can be a beneficial indicator in this respect. Such methods can be useful to determine whether FIBs are present around pre-harvest fresh produce, for example, indicating whether the product is safe for consumption. Furthermore, these assays can be used as part of the pre-season planning to determine which areas are safe for growing (i.e., at all or certain crops). Accordingly low-cost, rapid and easy to use methods for monitoring fecal contamination using the LAMP assays hereof are provided.

In certain embodiments, a method for assessing and/or monitoring fecal contamination comprises: providing at least one LAMP primer set that targets a DNA fragment of FIB in a sample, wherein the assay allows for single-step identification of the presence or absence of the FIB in the sample; obtaining a sample from a target (e.g., an unplanted field or a planted field prior to harvest); combining the sample with the at least one LAMP primer set into a mixture; heating the combination to initiate amplification of the targeted DNA fragment; and detecting a visual result in the heated combination indicative of the presence or absence of the FIB in the sample. Because FIBs are known to be highly abundant in fecal matter and are considered a reliable indicator of fecal contamination in water sources, the presence of FIBs can be used as an indirect measure of the presence of fecal matter and, thus, the potentially harmful pathogens associated with it. Bacteroidales, in particular, are not likely to occur in non-fecal sources and, as such, the methods hereof are highly accurate in identifying fecal contamination. Accordingly, in certain embodiments, where detection of a visual result indicates the presence of the targeted FIB in the sample, this is also indicative of the presence of a foodbome pathogen in the sample (i.e., that the fresh produce or field is “high risk”). Conversely, the absence of the targeted FIB in the sample can be indicative of the absence of a foodbome pathogen in the sample (i.e., the fresh produce or field is “low risk”).

The LAMP assay used in the method can be any assay described herein. At least one LAMP primer set can be any of the LAMP primer sets described herein. In certain embodiments, the FIB is Bacteroidales and the at least one LAMP primer set comprises primers of SEQ ID NOS: 4-9.

The target can comprise a field. The target can comprise a planted field (e.g., pre-harvest). The target can comprise an unplanted field (e.g., pre-plant and/or prior to the growing season). The sample can comprise a plurality of samples collected from various locations across the field.

As FIBs and foodbome pathogens are both consistently present in fecal matter, detection of a visual result that is indicative of the presence of the targeted FIB in the sample can, in some embodiments, also be indicative of the presence of a foodbome pathogen in the sample. In certain embodiments, the visual result is indicative of the presence of the targeted FIB in the sample where the concentration of the targeted FIB on a collection surface area (e.g., a leaf or a collection flag surface from which the sample is collected) is greater than the LoD of the assay. In certain embodiments, the LoD of the assay is about 17 copies of FIB per cm² (such as 17 copies/cm²) surface area of a collection surface from which the sample was obtained. In certain embodiments, the LoD of the assay is about 20 copies/cm² (such as 20 copies/cm²) surface area of a collection surface from which the sample was obtained. In certain embodiments, the LoD of the assay is about 100 copies/cm² (such as 100 copies/cm²) surface area of a collection surface from which the sample was obtained. In certain embodiments, the LoD of the assay is about 1250 copies/cm² (such as 1250 copies/cm²) surface area of a collection surface from which the sample was obtained. In certain embodiments, the LoD of the assay is about 10³ copies/cm² (such as 10³ copies/cm²) surface area of a collection surface from which the sample was obtained. In certain embodiments, the LoD of the assay is about 10³-10⁴ copies/cm² (such as 10³-10⁴ copies/cm²) surface area of a collection surface from which the sample was obtained.

In certain embodiments, the method further comprises identifying the target (e.g., a fresh produce crop, a field, or an unplanted field) as “high-risk” if the visual result equates with a surface concentration of the target FIB at or about 4 orders of magnitude greater than a “low-risk” value. The “low-risk” value can be at or about 2 copies/cm² of surface area of a collection surface from which the sample was obtained. The “low-risk” value can be at or about 10 copies/cm² of surface area of a collection surface from which the sample was obtained. The “low-risk” value can be less than 17 copies/cm² of surface area of a collection surface from which the sample was obtained (e.g., 16 copies/cm², 15 copies/cm², 15 copies/cm², 14 copies/cm², 13 copies/cm², 12 copies/cm², 11 copies/cm², 10 copies/cm², 9 copies/cm², 8 copies/cm², 7 copies/cm², 6 copies/cm², 5 copies/cm², 4 copies/cm², 3 copies/cm², 2 copies/cm², 1 copies/cm², or less than 1 copies/cm²).

In certain embodiments, if the visual result indicates the presence of the targeted FIB in the sample, the method further comprises destroying a crop planted in the field. If the visual result indicates the absence of the targeted FIB in the sample, the method can further comprise harvesting a crop planted in the field. Where the field is not yet planted, if the visual result indicates the presence of the targeted FIB (i.e., indicative of fecal contamination or that the field is “high-risk”), the method can further comprise planting crops in the field that are not intended for human raw consumption (e.g., corn or other crops that are typically subjected to heat or other treatments prior to consumption).

The ability to quickly and accurately quantify FIB in a sample not only provides a practical method that farmers can use to test their crops and/or fields for fecal contamination, but also allows for the determination of baseline concentration of FIB in commercial fields committed to safe production standards. These baseline standards have significant practical implications for fecal contamination management in the fresh produce industry including facilitating the interpretation of fecal contamination levels from a measured FIB concentration (e.g., meaningfully being able to determine if an acceptable or “safe” level of Bacteroidales, for example, is present, or if there is a fecal contamination issue with a crop) and/or assisting fresh produce growers to determine site-specific risk and their decision-making process regarding the microbial safety of fresh produce.

Clear risk thresholds between the presence of Bacteroidales and the correlative presence of foodborne pathogens have not been conventionally established. Although it is known that Salmonella spp. could be at a concentration of 10³-10⁴ bacteria/g of stool and E. coli 0157 could be less than 100 CFU/g of stool, how these concentrations relate to how much feces, pathogens, and Bacteroidales are present on fresh produce has conventionally remained undetermined. LeJeune et al., Sensitivity of Escherichia coli 0157 detection in bovine feces assessed by Broth enrichment followed by immunomagnetic separation and direct plating methodologies, J Clinical Microbiology 44(3): 872-875 (2006); Ohta et al., Quantitative dynamics of Salmonella and E. coli in feces of feedlot cattle treated with ceftiofur and chlortetracycline, PLoS One 14(12) (2019).

FIB such as Escherichia coli, Enterococcus faecalis, and Bacteroidales, have been used to assess possible fecal contamination in fresh produce. FIBs can serve as a quantitative marker in each farm, not only to assess the risk of contamination based on the farm's unique combination of environmental risk variables, but also to track the source and resolve fecal contamination. One of the reasons this methodology has been heretofore unavailable, however, is that the levels of Bacteroidales, in particular, that are naturally present in the environment of various fresh produce operations remained undetermined. While the presence of Bacteroidales indicates fecal contamination, fecal contamination is not always associated with the presence of enteric pathogens.

FIBs are normally present in much higher concentrations than any of the pathogens and are also more constantly detected in stool samples, as compared to pathogens. Korajkic et al., Relationships between Microbial Indicators and Pathogens in Recreational Water Settings, International J Environmental Research & Public Health 15: 2842 (2018). As a result, focusing exclusively on pathogen screening could deliver a false-negative result and conceal the fact there is a high risk of fecal exposure in the field. If an extraordinarily high level of Bacteroidales was detected in the field, for example, regardless of the presence or absence of pathogens, it implies that the field has been exposed to serious fecal contamination and the grower must act immediately to remedy the exposure.

Methods of microbial source tracking are also provided. The LAMP assays hereof can be used to not only identify the presence or absence of FIBs in a sample, but the LAMP assays can also be used to identify the source(s) of the contamination (e.g., the species from which the contamination originated).

A method of microbial source tracking can comprise. providing a first LAMP primer set that targets a DNA fragment of a first targeted FIB in a sample; obtaining a sample from a target; combining the sample and first LAMP primer set into a mixture; heating the combination to initiate amplification of the targeted DNA fragment; and detecting a visual result in the heated combination indicative of the presence or absence of the first targeted FIB in the sample. The first targeted FIB can be an FIB of a first species and the first LAMP primer set ca be species-specific to the first species.

The LAMP primer set can be any of the LAMP primer sets described herein. In certain embodiments, the LAMP primer set comprises primers comprising SEQ ID NOS: 4, 5, 6, 7, 8, and 9. In certain embodiments, the first LAMP primer set is coupled with a colorimetric reagent of a first color and the visual result indicative of the presence of the first targeted FIB in the sample (e.g., a positive result) comprises the first color.

The method can further comprise providing a second LAMP primer set that targets a DNA fragment of a second targeted FIB in the sample; combining the sample and the second LAMP primer set into a mixture; heating the combination to initiate amplification of the targeted DNA fragment; and detecting a visual result in the heated combination indicative of the presence or absence of the second FIB in the sample, wherein the second targeted FIB is an FIB of a second species and the second LAMP primer set is species-specific to the second species. In certain embodiments, the first primer set can comprise SEQ ID NOS: 23-25 and the second primer set can comprise SEQ ID NOS: 26-28, SEQ ID NOS: 29-31, and/or SEQ ID NOS: 32-34.

In certain embodiments, the method can further comprise tracking sources of contamination by using primer sets comprising host-associated 16S rRNA gene sequences.

The second LAMP primer set can be coupled with a colorimetric reagent of a second color and the visual result indicative of the presence of the second targeted FIB, for example, can comprise the second color. Using colorimetric reagents in this manner, the assays can simply indicate if contamination is present, if so, the species from which the contamination originated, and the results can easily be seen by the naked-eye.

The visual result can be provided in about 60 minutes or less (such as in 60 minutes or less) of initiating the heating step. The sample can be a bioaerosol sample. The target can be a field (planted or unplanted).

In certain embodiments, the target is a field and the method further comprises: collecting one or more collection flags from the field, wherein each collection flag comprises a film affixed to a support; and swabbing the sample of a surface of the film of each collection flag. The film of the collection flag can be a transparent film. The film of the collection flag can comprise a plastic. The collection flag can be encoded with a unique identifier indicative of the location in the field in which the collection flag was positioned. In such embodiments, the method can further comprise generating a map of the visual results by associating each visual result with the respective unique identifier of the collection flag from which the respective sample was obtained. The map can be a heat map.

Mapping these results can provide insight into contamination events; for example, animal (i.e., wildlife) intrusion, insanitary operations, and/or irrigation water contamination would all show up as hot spots on a heat map, indicating areas of potential vulnerability and/or sources of contamination to be addressed.

### EXAMPLES

The following examples illustrate certain specific embodiments of the present disclosure and are not meant to limit the scope of the claimed invention in any way.

### Example 1

### Materials & General Methods

Genomic DNA preparation and fecal DNA extraction. B. fragilis (ATCC® 25285™) was grown overnight (37° C., 4% H₂, 5% CO₂, 91% N₂, <20 ppm 02) in Chopped Meat Carbohydrate Broth (BD297307; BD, USA). Genomic DNA was extracted from B. fragilis with Purelink Genomic DNA Mini Kit (K182001; Invitrogen, USA) according to the manufacturer's protocol.

Stool samples (from cattle, swine, and poultry) were collected using a disposable utensil while steaming. The samples were transferred to sterilized 50 mL centrifuge tubes and were immediately stored in an icebox. Samples were mixed with 15% glycerol and stored at −80° C. until nucleic acid extraction. The human fecal matter was purchased from Lee Biosolutions (991-18; Lee Biosolutions, USA). The genomic DNA of human and animal stool samples were extracted with Fast DNA Stool Mini Kit (51604; QIAGEN, Germany) according to the manufacturer's protocol.

Quantitative Polymerase Chain Reaction (qPCR). The qPCR reaction was performed in a total volume of 25 μl, containing 12.5 μl 2× Luna® Universal Probe qPCR Master Mix (M3004; New England Biolabs, USA) (final concentration 1×), 1 μl of 10 μM forward and reverse primers (final concentration 0.4 μM) (Table 1 (Siefring et al., Improved realtime PCR assays for the detection of fecal indicator bacteria in surface waters with different instrument and reagent systems, J Water Health 6(2): 225-237 (2008)), 0.5 μl of 10 μM fluorescent probe (final concentration 0.2 μM) (Table 1), 9 μl nuclease-free water, and 1 μL of template or 1 μL of nuclease-free water for no template control (NTC).

TABLE 1
Sequences for qPCR primers used

| | | SEQ ID |
|---|---|---|
| Primer | Sequence (5′ - 3′) | NO: |
| GenBac3.FP | GGGGTTCTGAGAGGAAGGT | 1 |
| GenBac3.RP | CCGTCATCCTTCACGCTACT | 2 |
| GenBac3.Probe | CAATATTCCTCACTGCTGCC | 3 |
| | TCCCGTA | |

The qPCR reactions were performed on a qTOWER³ Real-Time Thermal Cycler (Analytik Jena, Germany), and the thermal cycling conditions were implemented using the following program: initial denaturation at 95° C. for 1 minute, followed by 45 cycles of 95° C. for 15 seconds, 55° C. for 15 seconds, and 60° C. for 30 seconds.

LAMP. Except where otherwise indicated, the LAMP reactions were performed using the designed primers (described in Example 2 below). The fluorometric LAMP reaction was performed in a total of 25 μl comprising 12.5 μL WarmStart LAMP 2× Master Mix (E1700; New England Biolabs, USA) (final concentration 1×). 0.5 μL Fluorescent dye 50× (B1700AVIAL; New England Biolabs, USA) (final concentration 1×), 2.5 μL 10×LAMP primer mix (16 μM FIP/BIP, 2 μM F3/B3, 4 μM LF/LB) (final concentration 1.6 μM FIP/BIP, 0.2 μM F3/B3, 0.4 μM LF/LB), 8.5 μl nuclease-free water, and 1 μL of template or 1 μL of nuclease-free water for NTC. The colorimetric LAMP reaction was performed in a total of 25 μl comprising 12.5 μL WarmStart© Colorimetric LAMP 2× Master Mix (M1800; New England Biolabs, USA) (final concentration 1×), 5 μL of 5 μM SYTO™ 9 Green Fluorescent Nucleic Acid Stain (S34854; Invitrogen, USA) (final concentration 1 μM), 2.5 μL 10×LAMP primer mix (16 μM FIP/BIP, 2 μM F3/B3, 4 μM LF/LB) (final concentration 1.6 μM FIP/BIP, 0.2 μM F3/B3, 0.4 μM LF/LB), 4 μl nuclease-free water, and 1 μL of template or 1 μL of nuclease-free water for NTC.

### Example 2

### Lamp Primer Design and Screening

Three sets of LAMP primers were designed based on the conservative region found via NCBI Multiple Sequence Alignment (Table 2). Briefly, multiple sequence alignment was performed using the NCBI Multiple Sequence Alignment Viewer (MSA). A conservative region on the Bacteroidales 16S ribosomal RNA gene was found by aligning the first 1000 hits of NCBI BLAST using the algorithm somewhat similar sequences (blastn). The LAMP primer set was designed based on the conservative region using PrimerExplorer V5 (publicly available software) with the default parameters (Table 2).

TABLE 2
Sequences for selected LAMP primer set targeting
Bacteroidales. The primer naming convention is
Host.bacteria.gene.primer_set#.type_of_primer

| | | SEQ ID |
|---|---|---|
| Primer | Sequence (5′ - 3′) | NO: |
| Universal.Bacteroidales.16S | TGCGGGTATCGAACAGGATT | 4 |
| rRNA.1.F3 | | |
| Universal.Bacteroidales.16S | GGTAAGGTTCCTCGCGTATC | 5 |
| rRNA.1.B3 | | |
| Universal.Bacteroidales.16S | TTAACGCTTTCGCTTGGCCACAGTAGTC | 6 |
| rRNA.1.FIP | CGCACGGTAAACG | |
| Universal.Bacteroidales.16S | GTACGCCGGCAACGGTGAAAACATGTTC | 7 |
| rRNA.1.BIP | CTCCGCTTGTG | |
| Universal.Bacteroidales.16S | GGCCGAACAGCGAGCAT | 8 |
| rRNA.1.LF | | |
| Universal.Bacteroidales.16S | CAAAGGAATTGACGGGGGC | 9 |
| rRNA.1.LB | | |
| Universal.Bacteroidales.16S | GATGGGGATGCGTTCCATT | 10 |
| rRNA.2.F3 | | |
| Universal.Bacteroidales.16S | CCATAGGGCAGTCATCCTTC | 11 |
| rRNA.2.B3 | | |
| Universal.Bacteroidales.16S | CCAATGTGGGGGACCTTCCTCGGTAACG | 12 |
| rRNA.2.FIP | GCCCACCTAGT | |
| Universal.Bacteroidales.16S | CACGGTCCAAACTCCTACGGGGCTACTT | 13 |
| rRNA.2.BIP | GGCTGGTTCAGG | |
| Universal.Bacteroidales.16S | TGCTACCTATCCCCAAGAC | 14 |
| rRNA.2.LF | | |
| Universal.Bacteroidales.16S | AGGAATATTGGTCAATGGGCG | 15 |
| rRNA.2.LB | | |
| Universal.Bacteroidales.16S | AGGATGAAGGTCCTACGGAT | 16 |
| rRNA.3.F3 | | |
| Universal.Bacteroidales.16S | AGCCCCAAACTTTCACAGC | 17 |
| rRNA.3.B3 | | |
| Universal.Bacteroidales.16S | GGCACGGAGTTAGCCGATGCGGGAATAA | 18 |
| rRNA.3.FIP | ACCCTCCCACG | |
| Universal.Bacteroidales.16S | GCAGCCGCGGTAATACGGAGCTTAACGA | 19 |
| rRNA.3.BIP | CCCGTCTGCG | |
| Universal.Bacteroidales.16S | TCATAAGGTACATACAAGCTCCCAC | 20 |
| rRNA.3.LF | | |
| Universal.Bacteroidales.16S | CCGGATTTATTGGGTTTAAAGGGA | 21 |
| IRNA.3.LB | | |

The LAMP primer set was tested with both fluorometric and colorimetric LAMP assays. 1 ng of B. fragilis pure culture DNA (176,975 copies) extract was used as the template for the primer screening in each case. NTC had 1 μL of nuclease-free water instead of B. fragilis DNA.

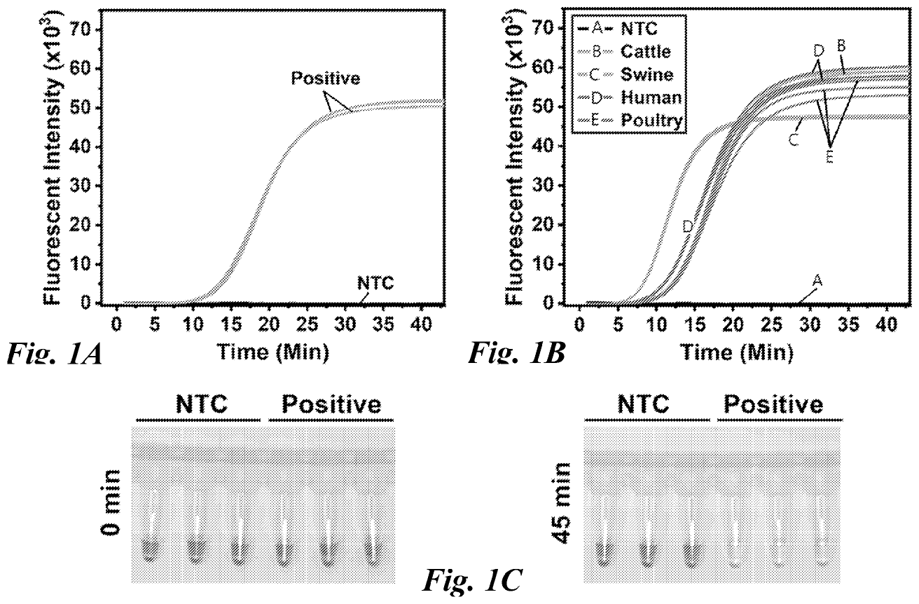

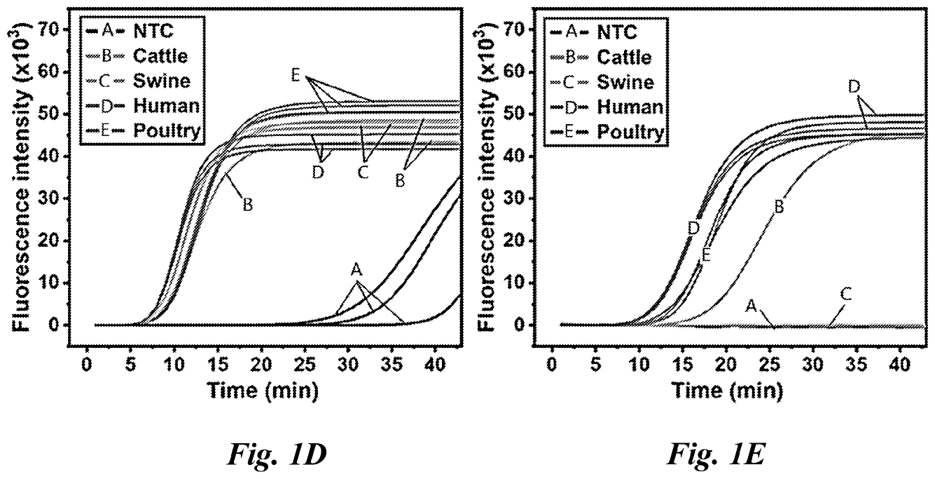

For the fluorometric LAMP assays, stool DNA extracts from different hosts were used (FIG. S3). For the first fluorometric study, 1 μL of B. fragilis DNA extract was added to the reaction mix to result in a final concentration 1 ng of total DNA (1×10⁶ copies of 16S rRNA) per reaction (FIG. 1A). For a second colorimetric study, 1 μL of stool DNA extract was added to the reaction mix to result in a final concentration 1 ng of total DNA per reaction (FIG. 1). Reactions had a final volume of 25 μL and used NEB WarmStart LAMP 2× Master Mix. Reactions were run on a qTower³ G at 65° C. with a ramp rate of 1° C./s.

In addition, a colorimetric (endpoint) LAMP assay was performed for the optimal primer set (FIG. 1C). 1 μL of B. fragilis DNA extract was added to the reaction mix to result in a final concentration 1 ng of total DNA per reaction. Before and after the reaction, samples were imaged via a flatbed scanner. The three samples on the left are NTC and the three samples on the right are positive samples. Colorimetric reactions were run with Anova Culinary Precision Cooker (ANTC01; Anova, USA) at 149° F. (65° C.). NTC: no template control where 1 μL of nuclease-free water was added to the reaction mix instead of DNA extract.

For all positive samples using Universal. Bacteroidales.16S rRNA.1 (i.e., a primer set comprising primers of SEQ ID NOS: 4, 5, 6, 7, 8, and 9 and otherwise identified herein as “Primer set 1”), both a fluorescence augment (FIGS. 1A and 1B) and a color change (FIG. 1C) were observed within 45 minutes.

Fluorometric and colorimetric data for all negative samples were consistent. No false positives were observed within all data. Primer set 1 was identified as the optimal primer set because it amplified the target Bacteroidales from all hosts without providing any false-positive amplification in the negative controls. Primer Set 1 was used for further testing.

### Example 3

### Sensitivity and Specificity

The sensitivity of LAMP and qPCR were measured using quantified B. fragilis DNA. The B. fragilis DNA was quantified using a Quant-iT™ PicoGreen™ dsDNA Assay (P7589; Invitrogen, USA) according to manufacturer's instructions. Both LAMP and pCR assays were performed as described in Example 1 above. Serial dilutions were made to determine the LoD of both LAMP and qPCR as described below. All reactions were done in triplicates.

More specifically, serially diluted (1000 copies/reactions to 1 copy/reaction) B. fragilis genomic DNA (1 μL) samples were added to reactions (24 μL reagents) in triplicates. The reactions were run in its respective thermal (cycling) condition. For the NTC, 1 μL of nuclease-free water was added to the reaction mix instead of resuspension. Reactions had a final volume of L and used NEB WarmStart LAMP 2× Master Mix (for LAMP studies) and NEB 2× Luna® Universal Probe qPCR Master Mix (for qPCR studies). Reactions for LAMP studies were run on a qTower³ G at 65° C. with a ramp rate of 1° C./s, and for qPCR studies, reactions were run on a qTower³ G with a ramp rate of 8° C./s.

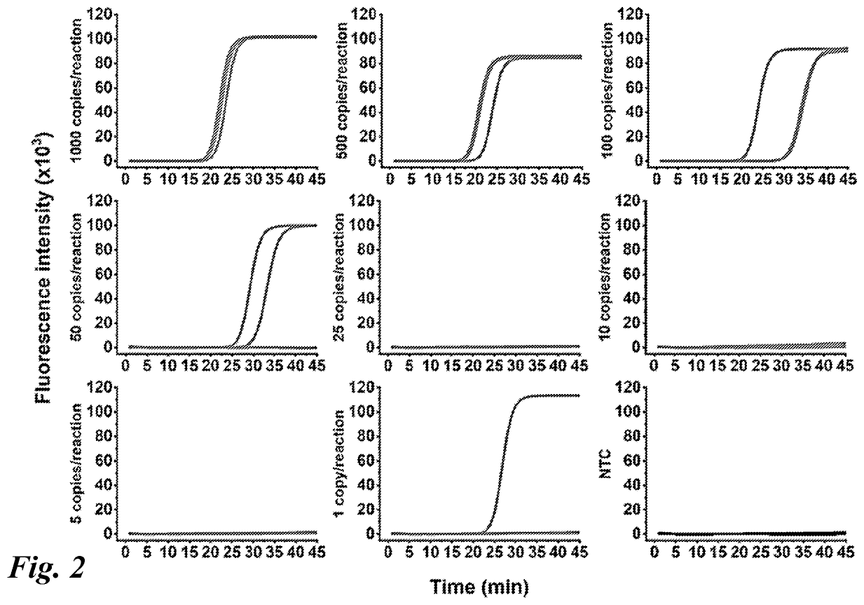

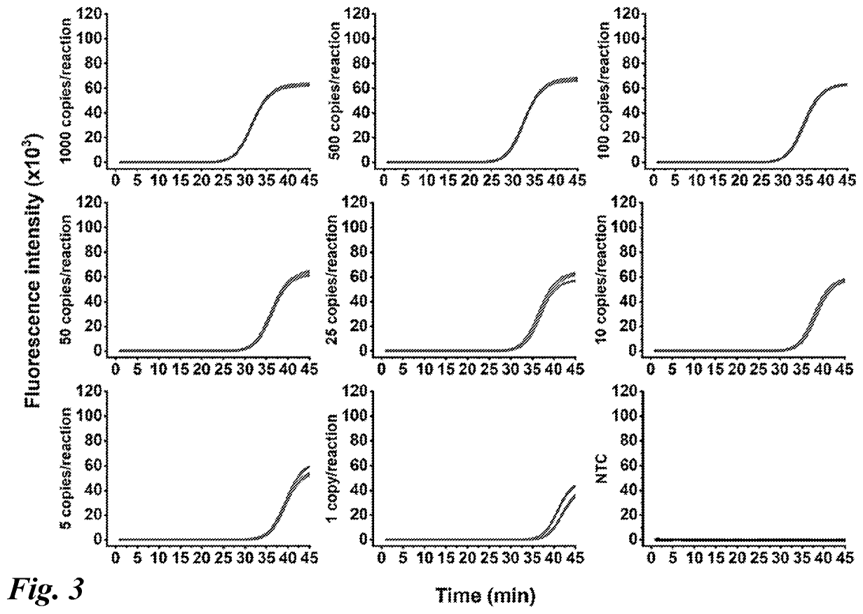

The fluorescence intensities were extracted for the 45-minute time point for both LAMP and qPCR reactions (FIGS. 2 and 3). Any amplification values that were greater than the highest background intensity (20% of the maximum reaction intensity) were considered successful amplifications.

The lowest DNA concentrations that had successful amplification for all three replicates of a given reaction were classified as the limit of detection (LoD) for the assay. Comparison of sensitivity between LAMP and qPCR showed that qPCR has a better LoD (1 copy/reaction) than LAMP (100 copies/reaction). The Tt values (time required for the fluorescent intensity to reach/exceed defined reaction threshold) for LAMP and Ct values (number of cycles required for fluorescent intensity to reach/exceed defined reaction threshold) for qPCR were calculated using software qPCRsoft 4.1 (baseline correction: 5, auto threshold) (Analytik Jena, Germany), and reported in Table 3.

TABLE 3
Limit of detection characterization of the assay. The Tt/Ct
value (in minutes) of each reaction is reported below.

| DNA | | | | | | |
|---|---|---|---|---|---|---|
| concentration | Bacteroidales | | | | | |
| (copies/reaction) | LAMP (Primer Set 1) | | | qPCR (GenBac3) | | |
| 1000 | 18.23 | 18.75 | 19.64 | 24.12 | 24.10 | 24.04 |
| 500 | 20.30 | 17.35 | 17.05 | 24.73 | 25.02 | 25.03 |
| 100 | 29.28 | 29.62 | 20.02 | 27.33 | 27.33 | 27.34 |
| 50 | 25.05 | 28.45 | No Tt | 28.34 | 28.89 | 28.48 |
| 25 | No Tt | No Tt | No Tt | 29.66 | 29.71 | 29.08 |
| 10 | No Tt | No Tt | No Tt | 31.21 | 30.74 | 31.33 |
| 5 | No Tt | No Tt | No Tt | 31.87 | 32.39 | 32.34 |
| 1 | No Tt | 22.59 | No Tt | 34.75 | 35.95 | 35.89 |
| 0 | No Tt | No Tt | No Tt | No Ct | No Ct | No Ct |

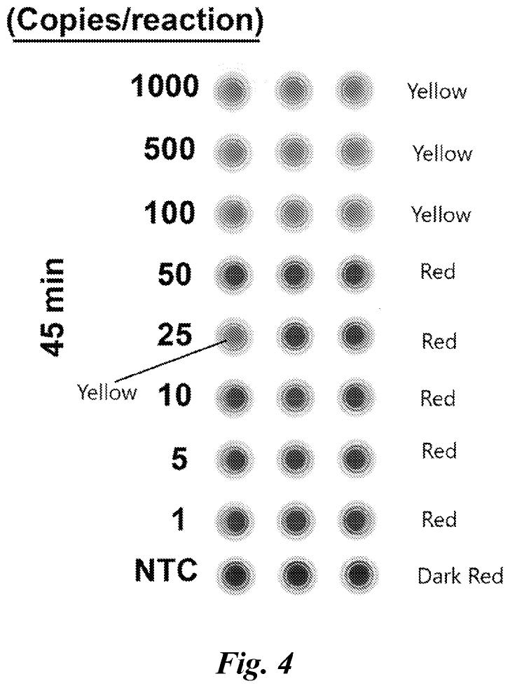

The same LoD experiment was repeated with colorimetric LAMP assay using the Anova Culinary Precision Cooker setup at the temperature of 149° F. (65° C.). Serially diluted (1000 copies/reaction to 1 copy/reaction) samples of B. fragilis genomic DNA (1 μL) were added to reactions (24 μL reagents) in triplicates. NTC indicates no template control where 1 μL of nuclease-free water was added to the reaction mix instead of resuspension. Reactions had a final volume of 25 μL and used NEB 2× colorimetric master. Reactions were run with a 12-quart container (B07RM787V2; Amazon, USA) filled with ultrapure water and an Anova Culinary Precision Cooker (ANTC01; Anova, USA) set to 149° F. (65° C.). The colorimetric LAMP assay showed the same LoD as the fluorescent LAMP assay (FIG. 4).

In-silico specificity studies were also conducted to verify the conservation of the LAMP primers with specific taxa of interest and to predict the cross-reactivity of the primer set. More specifically, these analyses checked whether the LAMP primers would react with seven bacterial species known to be associated with lettuce leaves surface microbiota based on a previous report and the NCBI strain isolation source. Rastogi et al., Leaf microbiota in an agroecosystem: spatiotemporal variation in bacterial community composition on field-grown lettuce, ISME J 6(10): 1812-1822 (2012).

In-silico sequence-specificity analysis were conducted by performing a BLAST search of the sequence LAMP primers spanned on (from the 5′ end of F3 to the 3′ end of complimentary B3) against sequences available in NCBI Nucleotide database for the specific taxon of interest. The parameters and results of the BLAST are shown in Table 4. The identities of the best BLAST hit were calculated by the number of nucleotide matches (including all N) divided by the total length of the sequence (213 base pairs).

TABLE 4
BLAST parameters used during in-silico analysis.

| Parameter | Value |
|---|---|
| Algorithm | blastn |
| Database | Nucleotide collection (nr/nt) |
| Entrez query | txid{TaxonID}[ORGN] |
| Expect threshold | 0.05 |
| Word size | 11 |
| Max target sequences | 1000 |
| Match/Mistmatch Score | 2, −3 |
| Gap existence/extension | 5, 2 |

Table 5 shows the overall sequence identity calculated by computing the maximum sequence identity of all hits for a single primer against an individual organism. Some cross-species similarity was excepted as the LAMP primers were designed based on the 16S ribosomal RNA gene, which is a highly conserved gene among diverse bacteria species. The in-silico sequence identity study revealed that the sequence identity rate is <50% for the seven microorganisms tested. Thus, the results suggest that these targets will not significantly cross-react with the primer set and were in agreement with the experimentally tested greenhouse controls, where amplification was not observed.

TABLE 5
Results from the in-silico sequence
identity analysis for Primer Set 1.

| Taxon | TXID | Identities |
|---|---|---|
| Bacteroidales | 171549 | 99.06% |
| Xanthomonas | 338 | 43.19% |
| Escherichia coli | 562 | 26.76% |
| Pantoea agglomerans | 549 | 26.76% |
| Bacillus subtilis | 1423 | 26.76% |
| Salmonella | 590 | 26.29% |
| Pseudomonas fluorescens | 294 | 26.29% |
| Massilia | 149698 | 26.29% |

In-silico sequence identity analyses were conducted by performing a BLAST search of the sequence LAMP primers spanned on (from the 5′ end of F3 to the 3′ end of complimentary B3) against sequences available in the NCBI Nucleotide database for the specific taxa. The nucleotide sequence of the sections that do not have a LAMP primer were converted to “N” indicating any nucleotide is acceptable. The parameters and results of the BLAST are shown in Table 3. The identities of the best BLAST hit were calculated by the number of nucleotide matches (including all N) divided by the total length of the sequence (213 base pairs).

### Example 4

### Host Inclusivity of the LAMP Assay

Stool DNA extract from four hosts (cattle, swine, poultry, and human) were used to test the host inclusivity of the LAMP assay. The stool extracts were diluted to 1 ng/μL and were used as the template for this study.

Briefly, stool DNA extracts from the four hosts (cattle, swine, poultry, humans) were spiked in LAMP reactions with Primer Set 1 to simulate different sources of fecal contamination. The LAMP assay detected Bacteroidales from all hosts' stool DNA extractions in 15 minutes with comparable Tt values (FIG. 1B). The detection in swine samples was slightly faster. There was no amplification in the negative controls.

The experiment indicates that the LAMP primer has high inclusivity among Bacteroidales from different hosts.

### Example 5

### Measurement of FIB in Leafy Greens and Collection Flags

The detection of artificially contaminated leafy greens was evaluated. Romaine lettuce (B01N5NG0OY; Amazon, USA) was grown (SF Gate Contributor, 2020) in a greenhouse (20° C.) at Purdue University. The mature lettuce (˜60 days) was placed around animal feeding operation facilities in Indiana, USA.

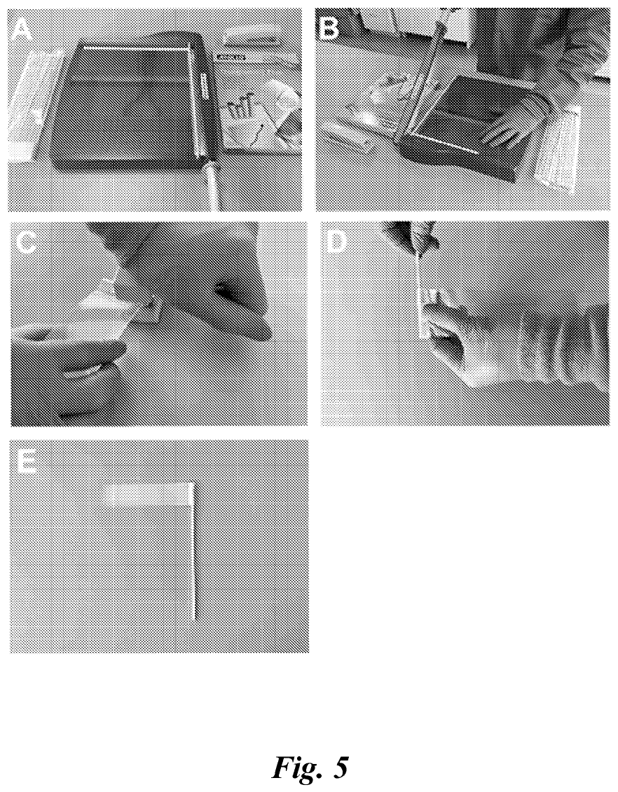

Collection flags were placed next to the lettuce plants. The collection flags were assembled using bamboo skewers (29.8 cm), transparent film (Apollo Plain Paper Copier Transparency Film), a stapler, and a paper-cutter. The transparent film was pre-cut into 5 cm×30 cm strips. Four pieces of the film were stapled together at the edge to form a loop. A bamboo skewer was inserted through the loop to make a collection flag. FIG. 5 illustrates the fabrication procedure.

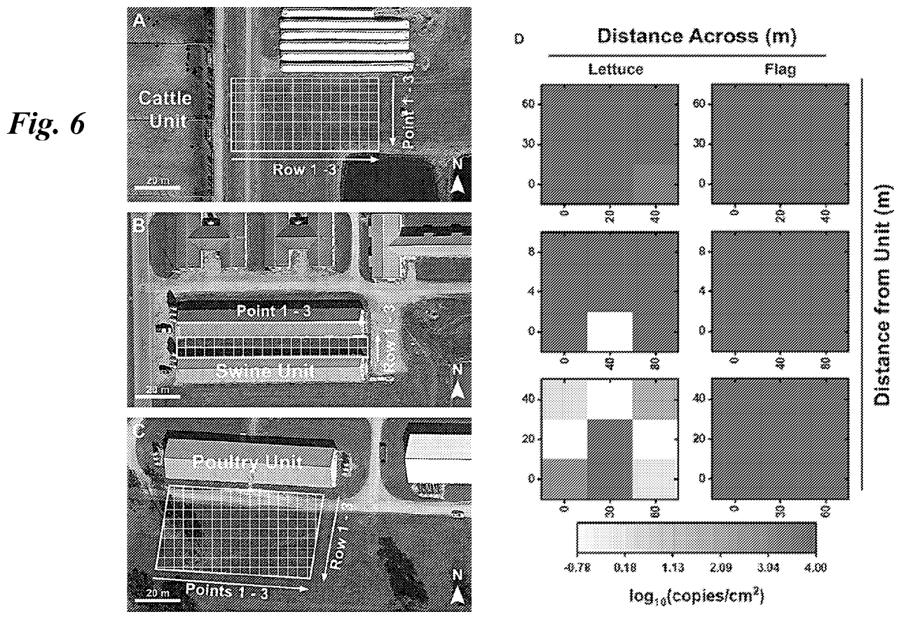

Ten plants and ten collection flags (per spot) were placed at three different distances (distance varies circumstantially due to the availability of space around animal units) away from each animal operation facility, with three replicates in each row (FIG. 6). Both the plants and collection flags were encoded with a unique identifier and the location associated with the plant/flag's identifier was recorded.

A group of ten lettuce and ten collection flags were placed in the greenhouse, which served as the negative control. After 7 days, all lettuce and collection flags were collected. Following United States Food and Drug Administration (FDA) Bacteriological Analytical Manual (BAM) for isolating specific pathogens from fresh vegetable samples, 25 g lettuce (approximately four leaves) or four pieces of transparency films were swabbed using a wet polyester-tipped swab (263000, BD BBL, USA). Each swab was resuspended in 200 μL molecular biology grade water. The resuspension was used for qPCR and LAMP assays (in lab and in the field).

The resuspended samples were used directly for molecular amplification assays. More specifically, 1 μL of resuspension was added to the reaction mix. Reactions had a final volume of 25 μL and used NEB WarmStart LAMP 2× Master Mix. Reactions were run on a qTower³ G at 65° C. with a ramp rate of 1° C./s.

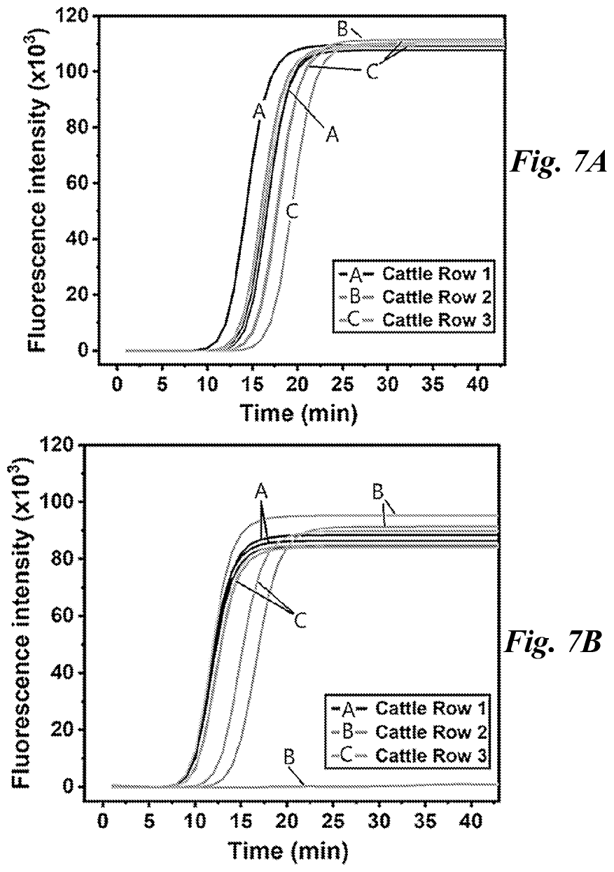

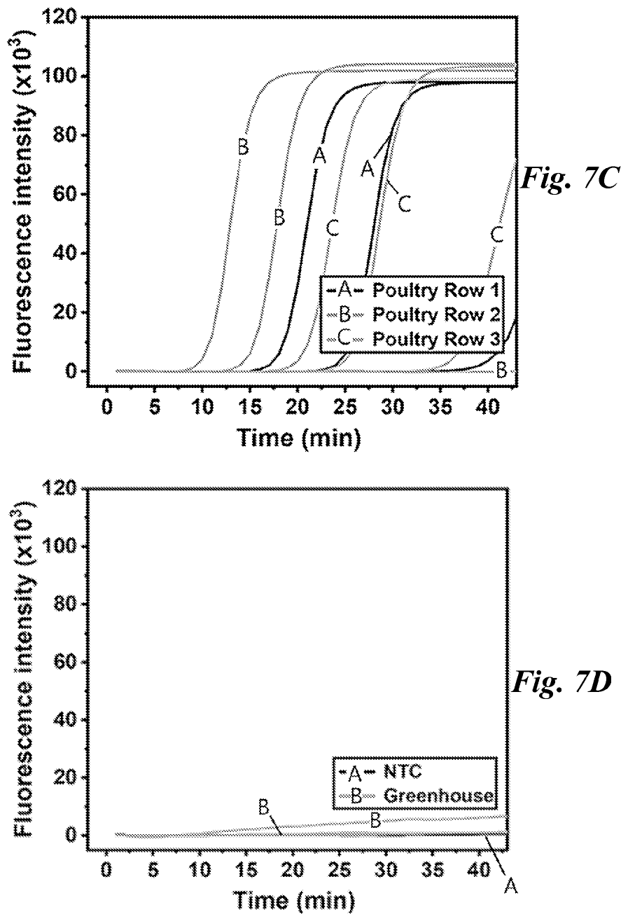

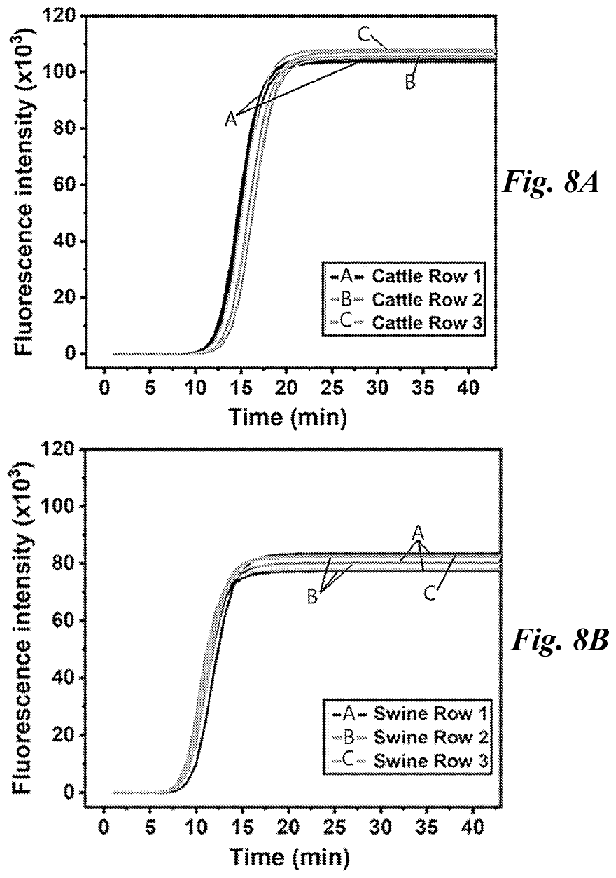

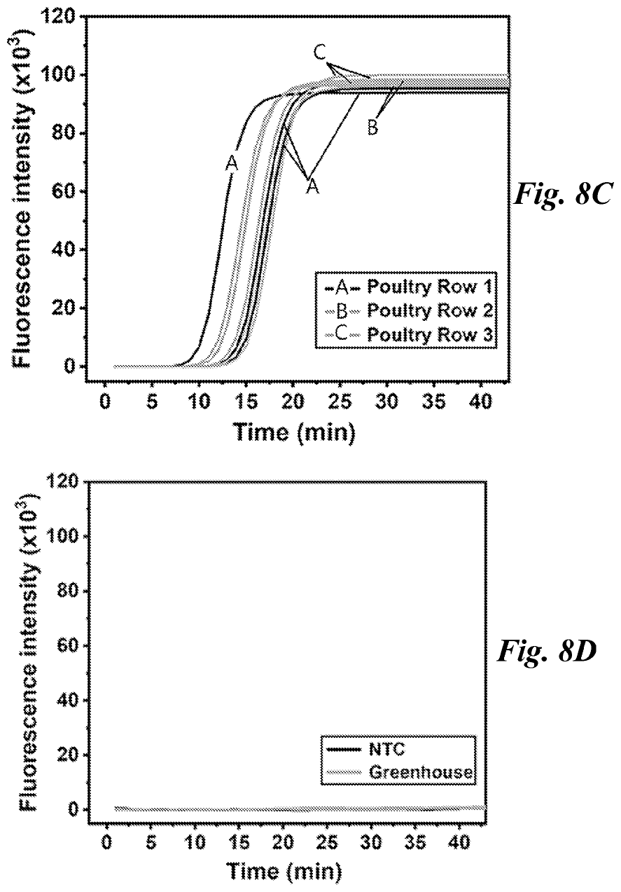

FIGS. 7A-7D and 8A-8D show the fluorometric LAMP data using swabs from lettuce leaves and collection flags, respectively. NTC indicates no template control where 1 μL of nuclease-free water was added to the reaction mix instead of resuspension solution.

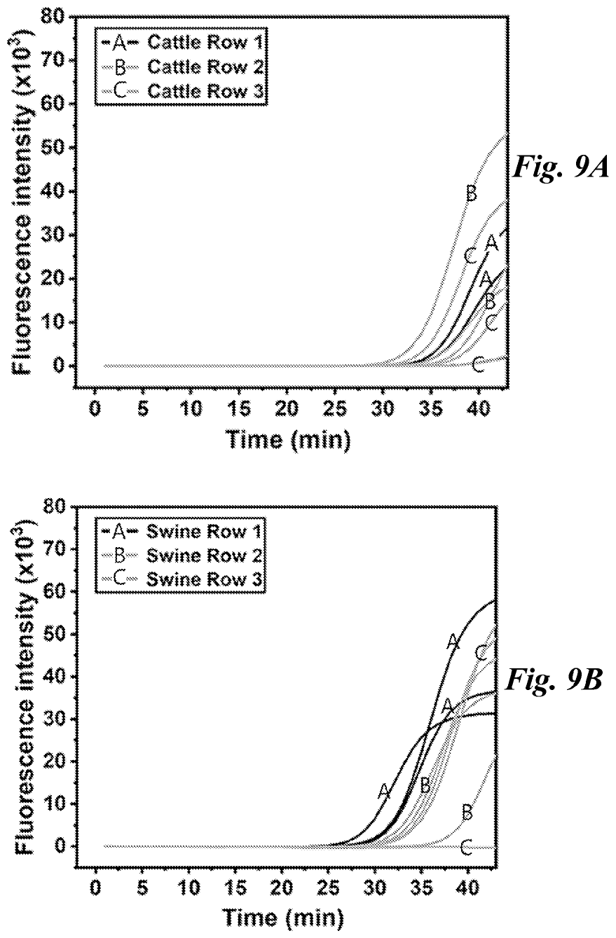

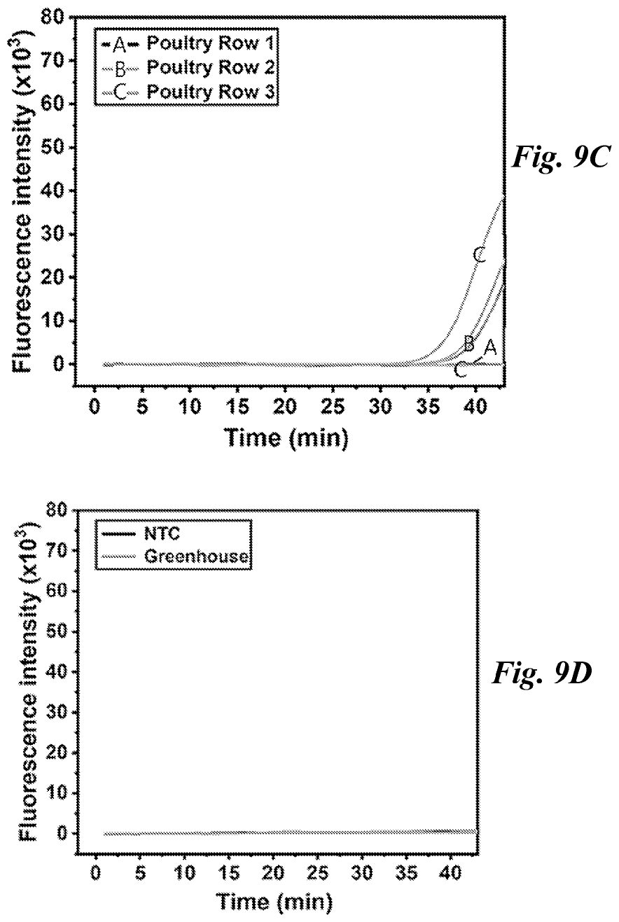

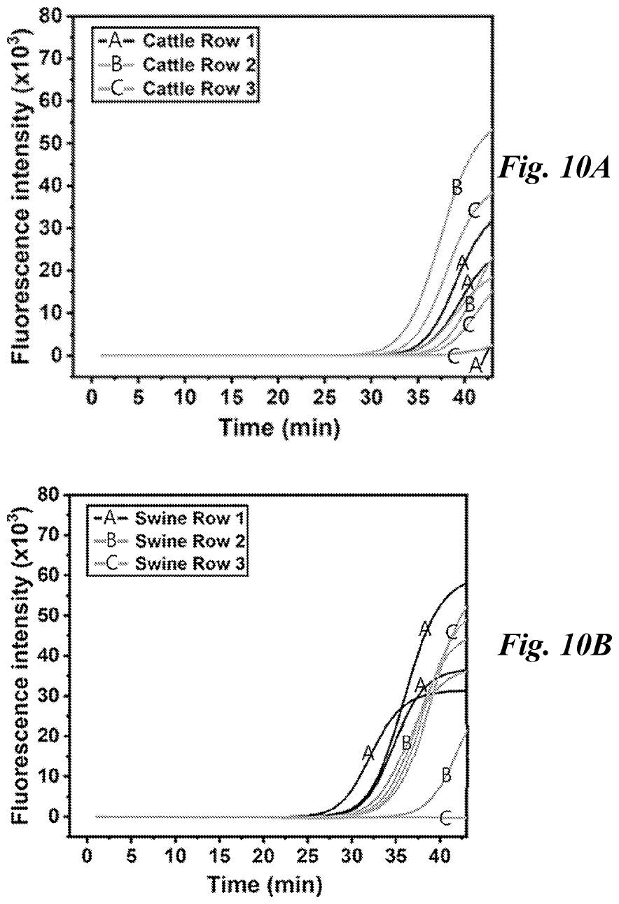

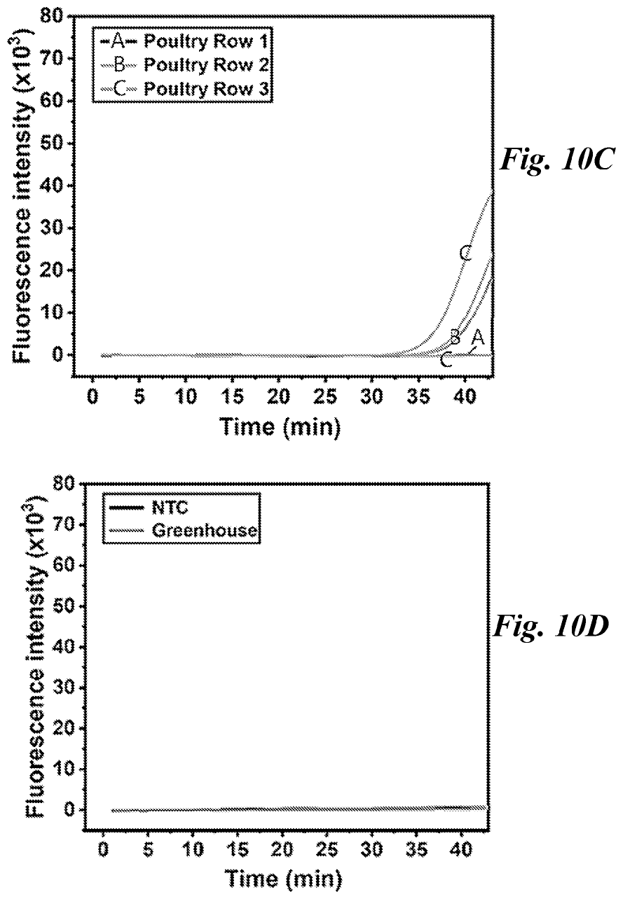

To confirm the results, qPCR was performed on the same samples (FIGS. 9A-9D and 10A-10D). Both outcomes appeared to be similar. Bacteroidales were not detected in the negative control group, indicating that neither the lettuce nor the collection flag samples were naturally contaminated with Bacteroidales or contaminated during the handling process.

It was also demonstrated that collection flag samples (FIGS. 8A-8D and 10A-10D) have higher consistency than lettuce swab samples (FIGS. 7A-7D and 9A-9D). Some of the swab samples from lettuce placed next to animal units did not amplify, and the amplification curves had high variability in the time-to-amplification. This could be due to the rough foliage topography, which makes consistent swabbing challenging. Thus, collection flags were used for on-site assay characterization studies.

### Example 6

### LAMP Assay Deployed On-Site

LAMP reactions were prepared in individual domed PCR tubes (AB0337; Thermo Fisher, USA) using a primer set comprising SEQ ID NOS: 4-9. The LAMP reactions were performed in a total of 25 μl comprising 12.5 μL WarmStart@Colorimetric LAMP 2× Master Mix (M1800; New England Biolabs, USA) (final concentration 1×), 2.5 μL 10×LAMP primer mix (16 μM FIP/BIP, 2 μM F3/B3, 4 μM LF/LB) (final concentration 1.6 μM FIP/BIP, 0.2 μM F3/B3, 0.4 μM LF/LB), 9 μl nuclease-free water, and 1 μL of resuspension or 1 μL of nuclease-free water for NTC.

For the on-site experiment, the reagents were prepared in the lab, and the addition of sample was done on-site using a 0.5-10 μL single-channel pipette (3123000020; Eppendorf, Germany) with no additional measures to avoid contamination. The experiment on-site happened no more than 30 minutes after swabbing the sample from collection flags.

Time-lapse video of the tubes was taken from 0 to 60 minutes using a HERO8 Black digital camera (GoPro, USA). Endpoint images of the tubes were taken at 0 and 60 minutes using a Sony Alpha a7II mirrorless digital camera (B00R1P93SC, Amazon, USA). All images obtained were adjusted by using the white balance tool on Adobe Lightroom to obtain a relatively uniform and consistent background.

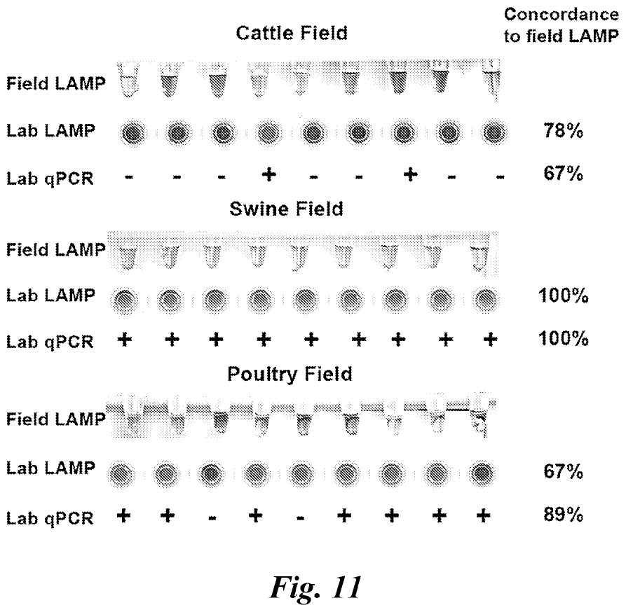

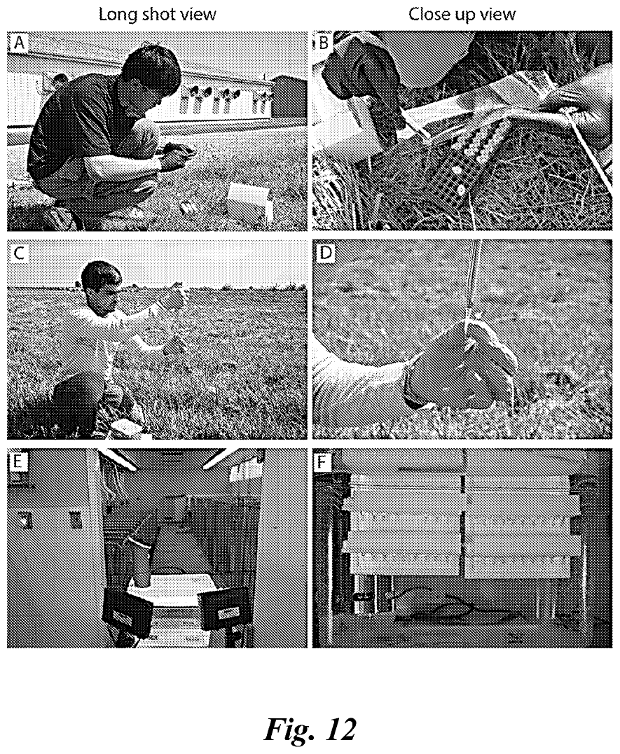

The collection flags were placed around the animal operation facilities (cattle, swine, poultry) for a period of seven days and LAMP assay was conducted on the seventh day. All samples, including the positive control (1 μL of 1 ng/μL B. fragilis gDNA) and no template control (1 μL of purified bottled drinking water), were added on-site without any additional measures to avoid contamination (FIGS. 11 and 12). More specifically, 1 μL of swab resuspension was added to the reaction mix. Reactions had a final volume of 25 μL and were run in the individual domed PCR tubes. A 12-quart container (B07RM787V2; Amazon, USA) was filled with bottled drinking water and an Anova Culinary Precision Cooker (ANTCOT; Anova, USA) set to 149° F. (65° C.) was attached as reported previously. Pascual-Garrigos et al., On-farm colorimetric detection of Pasteurella multocida, In: Mannheimia Haemolytica, and Histophilus Somni in Crude Bovine Nasal Samples 52: 126 (2021). The tubes were submerged in the water using a PCR tube holder designed and 3D-printed in-lab with a Form 3B 3D printer (Formlabs, MA) using high-temperature resin v2. Pascual-Garrigos et al. (2021), supra. The tubes were removed from the water after 60 minutes.

The same assay, as well as a qPCR assay, were conducted the next day in a lab setting using the same samples (FIG. 11). The qPCR used NEB 2× Luna® Universal Probe qPCR Master Mix. Lab confirmations were run on a qTower³ G with a ramp rate of 1° C./s and 8° C./s for LAMP and qPCR, respectively. For the qPCR tests, any fluorescent values that were greater than the highest background intensity were considered positive (+) amplifications, conversely negative (−) amplifications.

The concordance observed between LAMP assays performed on the farm and in the lab was 78%, 100%, and 67% for cattle, swine, and poultry, respectively. The concordance observed between LAMP assays performed on the farm and qPCR in the lab was 67%, 100%, and 89% for cattle, swine, and poultry, respectively. The lack of consistency in the cattle and poultry samples appears to be due to the lower concentration of DNA in the resuspended solution as compared to the swine samples. In addition, some of the samples from the cattle unit showed positive amplification in the field, but not in the lab tests, which could be due to degradation of the sample during transportation. Pascual-Garrigos et al. (2021), supra.

### Example 7

### Baseline Measurement Materials and Methods

To apply Bacteroidales for monitoring fecal contamination in fresh produce production, a baseline study was conducted to survey the background concentration of Bacteroidales in “low-risk” fresh produce fields. Fecal contamination in fresh produce under real-world conditions was surveyed. The goal was to establish a baseline measurement of Bacteroidales in fields with low fecal contamination risk.

Sample Collection. Briefly, a total of 1,632 samples were collected from two romaine lettuce commercial fields in California's Salinas Valley at the time of harvesting between May 2021 and August 2021 over two growing seasons. Both production fields complied with safe production standards, therefore, the baseline determined reflects the Bacteroidales level in fresh produce fields with “low risk” fecal contamination.

The fields were labeled with row and column numbers with the distance between each row and column to be 6 meters. Samples were collected at the intersection of each row and column (approximately 100 sampling sites per acre of field). Two types of samples were collected at each sampling site: 1) 25 g of romaine lettuce leaf sample (approximately four leaves); and 2) a collection flag sample. The sample size for the romaine lettuce leaf sample was determined following FDA BAM for isolating specific pathogens from fresh vegetable samples (FDA, 2021).

The fabrication and deploying method for the collection flags were as previously described herein and as set forth in Wang et al., A loop-mediated isothermal amplification assay to detect Bacteroidales and assess risk of fecal contamination, Food Microbiology 110: 104173 (2023). Briefly, the collection flags were assembled with a bamboo skewer (29.8 cm) and a piece of transparent film pre-cut into 7.62×21.59 cm (3×8.5 inches) strips. One collection flag was placed at each sampling site 7 days before the sample collection.

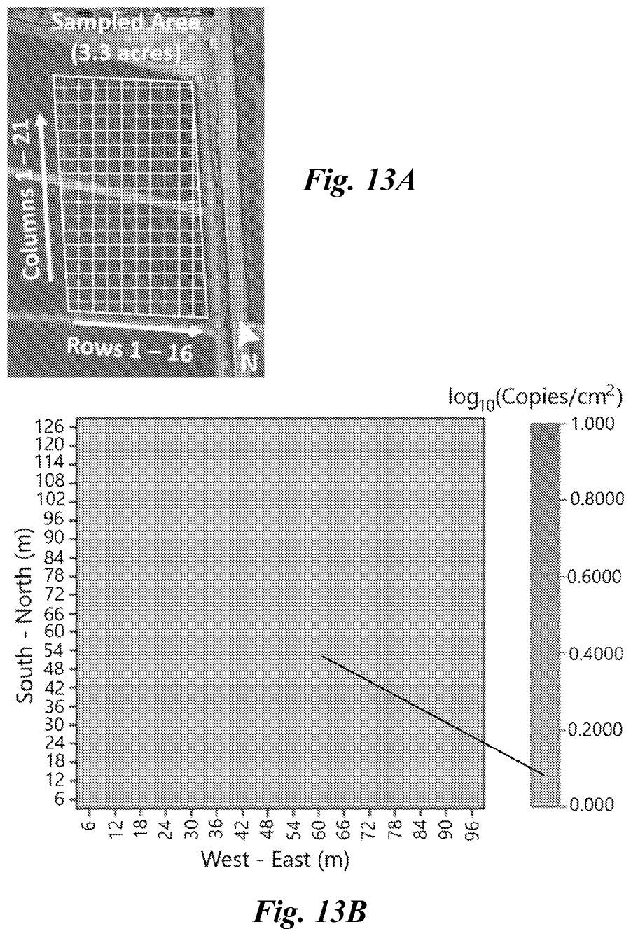

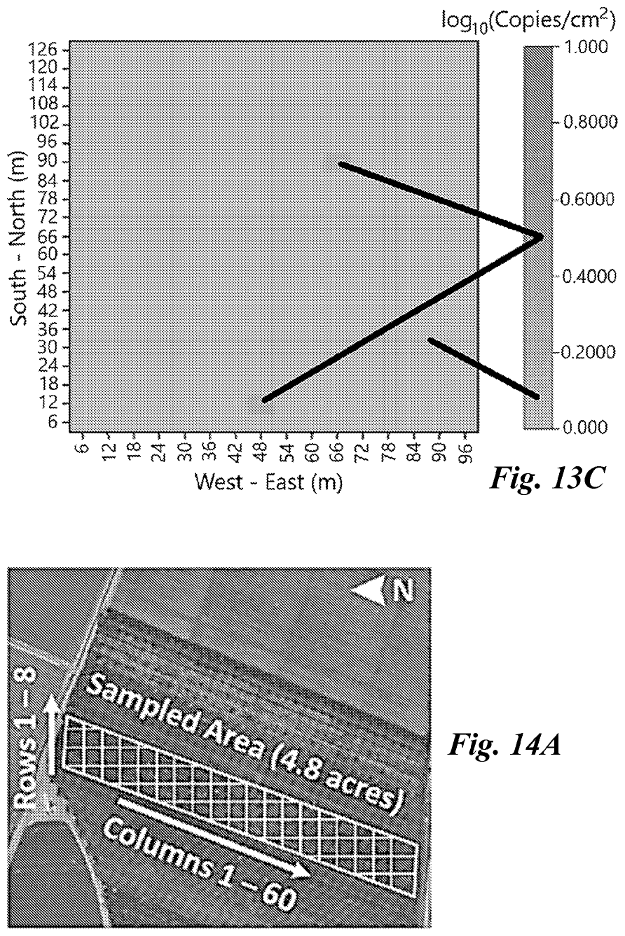

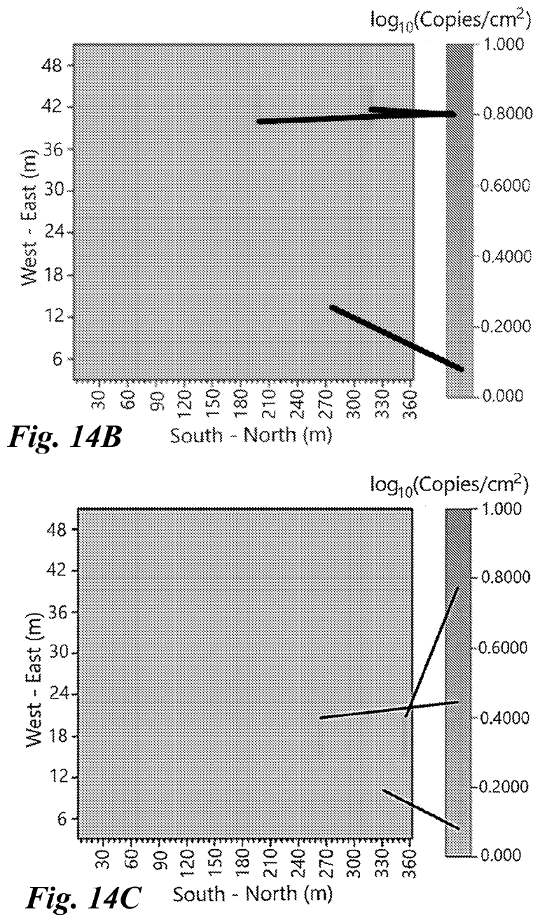

During the first growing season (May 2021), 336 romaine lettuce leaf samples and 336 collection flag samples were collected over a field size of 3.3 acres (16 rows, 21 columns) (FIG. 13A). In the second growing season (August 2021), 480 romaine lettuce leaf samples and 480 collection flag samples were collected over a field size of 4.8 acres (8 rows, 60 columns) (FIG. 14A). The samples were collected by individuals wearing Tyvek suits, gloves, and masks to avoid contaminating the samples. Additionally, before collecting each sample, 70% ethanol was used to sanitize gloves and sleeves to avoid cross-contamination.

Each sample was placed in an individual, pre-labeled Ziploc resealable storage bag (B07NQVYCG3; Amazon, USA). The collected samples were kept on ice and shipped to West Lafayette, Indiana via FedEx Priority Overnight in a cooler box with ice packs. Following sample collection, the remaining lettuce in the experimental fields was destroyed in the field.

Sample processing. The samples were stored at 4° C. after arriving in Indiana. The romaine lettuce leaf samples were processed by a washing and filtering method modified from the FDA BAM for isolating specific pathogens from fresh vegetable samples. FDA (2021); Wang et al. (2023), supra. Briefly, 225 mL of ultrapure water (PURELAB flex, ELGA, USA) was added to the sealed bag (B07NQVYCG3; Amazon, USA) with 25 g of lettuce leaf sample. The bag was hand-shaken for 1 minute to elute any bacteria into the solution. The wash solution was then filtered using a 90 mm, 0.22 μm, cellulose acetate (CA) membrane (FBM090CA022; Filter-Bio, China). The filtered membrane was immersed into 1 mL of nuclease-free water inside a 2 mL centrifuge tube and the tube was vortexed at maximum speed for 1 minute. Finally, the tube was centrifuged at 10,000 rpm for 1 minute to recover the resuspension. The membrane was removed from the tube after centrifugation. Each collection flag was swabbed using a wet polyester-tipped swab (263000, BD BBL, USA) and was resuspended in 200 μL nuclease-free water. All samples were kept at −20° C. until the experiment.

Genomic DNA preparation. B. fragilis (ATCC® 25285™) was grown overnight (37° C., 4% H2, 5% CO₂, 91% N₂, <10 ppm 02) in Chopped Meat Carbohydrate Broth (BD297307; BD, USA). Genomic DNA was extracted from B. fragilis with Purelink Genomic DNA Mini Kit (K182001; Invitrogen, USA) according to the manufacturer's protocol. The extracted DNA product was quantified using Quant-iT™ PicoGreen™ dsDNA Assay Kit (P7589; Thermo Fisher, USA).

qPCR. The qPCR reactions were performed in a total volume of 20 μL, containing 10 μL 2× Luna® Universal Probe qPCR Master Mix (M3004; New England Biolabs, USA) (final concentration 1×), 0.8 μL of 10 μM forward primer (final concentration 0.4 μM), 0.8 μL of 10 μM reverse primer (final concentration 0.4 μM), 0.4 μL of 10 μM fluorescent probe (final concentration 0.2 μM) (Table 1), 7 μL nuclease-free water, and 1 μL of template or 1 μL of nuclease-free water for NTC. The resuspensions of both membrane and swab were directly used for qPCR assays without performing DNA extraction. The qPCR reactions were performed on a qTOWER³ Real-Time Thermal Cycler (Analytik Jena, Germany), and the thermal cycling conditions were implemented using the following program: initial denaturation at 95° C. for 1 minute, followed by 45 cycles of 95° C. for 15 seconds, 55° C. for 15 seconds, and 60° C. for 30 seconds.

Digital PCR (dPCR). The dPCR reactions were performed in a total volume of 12 μL, containing 3 μL 4× Probe PCR Master Mix (250102; Qiagen, USA) (final concentration 1×), 1.2 μL of 10× primer-probe mix (final concentration 1×, 0.8 μM forward primer, 0.8 μM reverse primer, 0.4 μM FAM probe), 2.8 μL nuclease-free water, and 5 μL of the template or 5 μL of nuclease-free water for NTC. 10× primer-probe mix is one of the host-specific qPCR primer-probe set in Table 6 (cattle-specific Bacteroidales (Shanks et al., Quantitative PCR for Detection and Enumeration of Genetic Markers of Bovine Fecal Pollution, Applied Environmental Microbiology 74: 745-752 (2008)), swine-specific Bacteroidales (Mieszkin et al., Estimation of Pig Fecal Contamination in a River Catchment by Real-Time PCR Using Two Pig-Specific Bacteroidales 16S rRNA Genetic Markers, Applied Environmental Microbiology 75: 3045-3054 (2009)), human-specific Bacteroidales (Bernhard & Field (2000), supra and Converse et al, Rapid QPCR-based assay for fecal Bacteroides spp. as a tool for assessing fecal contamination in recreational waters, Water Research 43(19): 4828-4837 (2009)), and poultry-specific Bacteroidales (Kobayashi et al., Effects of temperature and predator on the persistence of host-specific Bacteroides-Prevotella genetic markers in water, Water Science & Technology 67: 838-845 (2013)).

TABLE 6
Sequences for primers and probes

| | | | SEQ |
|---|---|---|---|
| Target | Primer | Sequence (5′ - 3′) | ID NO: |
| Universal | GenBac3.FP | GGGGTTCTGAGAGGAAGGT | 1 |
| Bacteroidales | GenBac3.RP | CCGTCATCCTTCACGCTACT | 2 |
| | GenBac3.Probe | /FAM/CAATATTCC/ZEN/TCACTGCTGCCTC | 22 |
| | | CCGTA/IABKFQ/ | |
| | | (a primer sequence labeled with | |
| | | a fluorescent dye (FAM), flanked | |
| | | by quencher molecules (ZEN and | |
| | | IABKFQ)) | |
| Cattle | CowM2.FP | CGGCCAAATACTCCTGATCGT | 23 |
| specific | CowM2.RP | GCTTGTTGCGTTCCTTGAGATAAT | 24 |
| Bacteroidales | CowM2.Probe | /FAM/AGGCACCTA/ZEN/TGTCCTTTACCTC | 25 |
| | | ATCAACTACAGACA/IABKFQ/ | |
| Pig specific | Pig-2-Bac41F | GCATGAATTTAGCTTGCTAAATTTGAT | 26 |
| Bacteroidales | Pig-2- | ACCTCATACGGTATTAATCCGC | 27 |
| | Bac163Rm | /FAM/TCCACGGGA/ZEN/TAGCC/IABKFQ/ | 28 |
| | Pig- | | |
| | 2Bac113MGB | | |
| Human | HF183F | ATCATGAGTTCACATGTCCG | 29 |
| specific | BFDRev | CGTAGGAGTTTGGACCGTGT | 30 |
| Bacteroidales | BFDFAM | /FAM/CTGAGAGGA/ZEN/AGGTCCCCCACAT | 31 |
| | | TGGA/IABKFQ/ | |
| Poultry | qCD362_F | AATATTGGTCAATGGGCGAGAG | 32 |
| specific | qCD464_R | CACGTAGTGTCCGTTATTCCCTTA | 33 |
| Bacteroidales | qCD394_P | /FAM/TCCTTCACG/ZEN/CTACTTGG/IABk | 34 |
| | | FQ/ | |

The dPCR reactions were performed in an 8.5K 96-well Nanoplate (250021; Qiagen, USA) on a 5-plex QIAcuity One digital PCR instrument (911021; Qiagen, USA). The thermal cycling conditions were implemented using the following program: initial denaturation at 95° C. for 2 minutes, followed by 40 cycles of 95° C. for 15 seconds, 55° C. for 15 seconds, and 60° C. for 30 seconds.

### Example 8

### Baseline Measurement Studies: Construction of qPCR Calibration Curve

To apply Bacteroidales for monitoring fecal contamination in fresh produce production, a baseline study was conducted to survey the background concentration of Bacteroidales in “low-risk” fresh produce fields. Fecal contamination in fresh produce under real-world conditions was surveyed. The goal was to establish a baseline standard to assess a concentration of FIB in “low-risk” fresh produce fields as compared to a concentration of FIB in a “high-risk” fresh produce field.

The fluorescence intensities were extracted for the 45-minute time point for qPCR reactions. Briefly, B. fragilis genomic DNA (1 μL) was added to reactions (19 μL reagents) in triplicate at different concentrations (serially diluted from 1000 copies/reaction to I copy/reaction). For NTC, 1 μL of nuclease-free water was added to the reaction mix instead of genomic DNA. The final volume of each reaction was 20 μL, and the master mix used was NEB 2× Luna® Universal Probe qPCR Master Mix.

Any fluorescent intensity values that were greater than the highest background intensity (20% of the maximum reaction intensity) were considered successful amplifications. The lowest DNA concentration that had successful amplification for all three replicates was classified as the LoD.

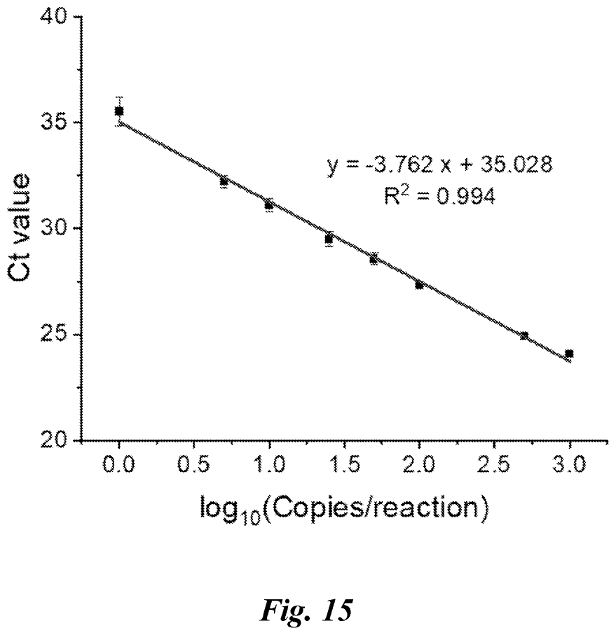

The qPCR showed a LoD of 1 copy/reaction. The Ct values (number of cycles (1 minute each) required for fluorescent intensity to reach/exceed defined reaction threshold) for qPCR were calculated using software qPCRsoft 4.1 (baseline correction: 5, auto threshold) (Analytik Jena, Germany), and reported in Table 7. Linear regression analysis was used to fit correlations between Ct values and log₁₀ (copies/reaction) (FIG. 15).

TABLE 7
LoD characterization of the assay

| DNA concentration | Bacteroidales | | |
|---|---|---|---|
| (copies/reaction) | qPCR (GenBac3) | | |
| 1000 | 24.12 | 24.10 | 24.04 |
| 500 | 24.73 | 25.02 | 25.03 |
| 100 | 27.33 | 27.33 | 27.34 |
| 50 | 28.34 | 28.89 | 28.48 |
| 25 | 29.66 | 29.71 | 29.08 |
| 10 | 31.21 | 30.74 | 31.33 |
| 5 | 31.87 | 32.39 | 32.34 |
| 1 | 34.75 | 35.95 | 35.89 |
| 0 | No Ct | No Ct | No Ct |

### Example 9

### Bacteroidales Concentration on Field-Grown Lettuce and Collection Flags

The processed samples from Example 7 were used for qPCR assays. The fluorescence intensities were extracted for the 45-min time point for qPCR reactions. The Ct values for qPCR were calculated using the software qPCRsoft 4.1 (baseline correction: 5, auto threshold) (Analytik Jena, Germany). The Ct value of each sample was then used to calculate the Bacteroidales concentration using the constructed calibration curve from Example 8.

To construct a fecal contamination risk evaluation map, the Bacteroidales concentration result of both types of samples (romaine lettuce leaf samples and collection flag samples) were normalized to log₁₀ (copies/cm²) (FIGS. 13A-14C).

During the May 2021 harvesting season, 672 samples (including 336 lettuce leaf samples and 336 collection flag samples) were collected from a sampled area of 3.3 acres. For most samples, Bacteroidales was not detected via qPCR; only 9 samples (leaf samples: R3C19, R11C16; flag samples: R2C20, R4C17, R6C1, R8C2, R10C2, R11C15, R16C6; R: row, C: column) returned a Bacteroidales concentration higher than 1 copy/reaction, which is the qPCR assay's LoD. The Bacteroidales concentrations of these samples are reported in Table 8.

During the August 2021 harvesting season, 960 samples (including 480 lettuce leaf samples and 480 collection flag samples) from a sampled area of 4.8 acres. Similar to the result from samples collected in May 2021, only 8 samples (leaf samples: R6C32, R7C33, R7C52, R7C53; flag samples: R3C44, R3C59, R5C1, R8C11; R: row, C: column) returned a Bacteroidales concentration higher than 1 copy/reaction (Table 8). As expected, the concentrations of Bacteroidales in both romaine lettuce commercial fields from two harvesting seasons were very low (0.62-2.00 copies/cm²).

TABLE 8
Samples selected for microbial source tracking

| Collection time | Sample type | Sample ID | Copies/cm² |
|---|---|---|---|
| May 2021 | Leaf sample | R3C19 | 0.88 |
| | | R11C16 | 0.87 |
| | Collection flag | R2C20 | 0.62 |
| | | R4C17 | 0.98 |
| | | R6C1 | 0.62 |
| | | R8C2 | 1.58 |
| | | R10C2 | 0.82 |
| | | R11C15 | 1.57 |
| | | R16C6 | 0.69 |
| August 2021 | Leaf sample | R6C32 | 0.82 |
| | | R7C33 | 1.32 |
| | | R7C52 | 1.28 |
| | | R7C53 | 1.67 |
| | Collection flag | R3C44 | 1.11 |
| | | R3C59 | 2.00 |
| | | R5C51 | 0.63 |
| | | R8C11 | 0.94 |

Accordingly, among the 1,632 samples collected, only 17 samples returned a concentration higher than 1 copy/reaction (about 1%). This finding is consistent with Bacteroidales not normally occurring in the environment, but a trace amount of Bacteroidales DNA being present in the field as a result of various reasons (e.g., organic fertilizer, residuals from previous contamination, etc.).

In previous studies, the present inventors determined the concentration of Bacteroidales in fields with “high risk” fecal contamination (e.g., those adjacent to animal feeding operations), with the concentration quantified at over 10⁴ copies/cm² (of Bacteroidales per cm²). Wang et al. (2023), supra. Compared to the present data where the highest concentration of Bacteroidales observed was 2 copies/cm². This supports there is a significant difference of about four orders of magnitude in the concentration of Bacteroidales between “high risk” and “low risk” of fecal contamination.

### Example 10

### Microbial Source Tracking

Samples that returned a concentration higher than 1 copy/reaction in Example 9 (17 samples) were selected for a microbial source tracking study. Each sample was tested with four different host-specific qPCR primer-probe sets (cattle-specific Bacteroidales, swine-specific Bacteroidales, human-specific Bacteroidales, and poultry-specific Bacteroidales) (Table 6).

Since the host-specific populations represent a small group within the general Bacteroidales population, a dPCR method was adopted for the microbial source tracking experiment to achieve a higher level of sensitivity. Bemhard and Field (2000), supra; Lamendella et al., Assessment of fecal pollution sources in a small northern-plains watershed using PCR and phylogenetic analyses of Bacteroidetes 16S rRNA gene, FEMS Microbiology & Ecology 59: 651-660 (2007); Mulbury et al., Determining lower limits of detection of digital PCR assays for cancer-related gene mutations, Biomol. Detect. Quantification 1: 8-22 (2014). dPCR is commonly used in environmental research and due to the inherent nature of dPCR, the assay has a high tolerance to biological inhibitors and has better performance on trace detection for a minority target. Milbury et al. (2014), supra; Perkins et al., Chapter Three—Droplet-Based Digital PCR: Application in Cancer Research, in: Makowski, G. S. (Ed.), Advances in Clinical Chemistry, Elsevier, pp. 43-91 (2017).

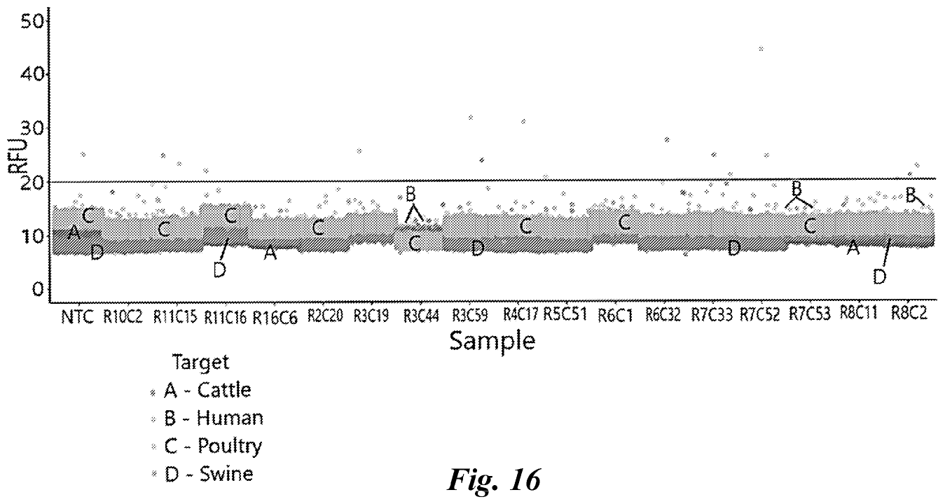

Following plate preparation, the system partitioned each sample into approximately 8,500 partitions, with approximately 8,300 valid counts. Each partition was individually sealed following 40 cycles of thermocycling. The plate was then imaged to count the number of positive/fluorescent partitions for each sample. The fluorescent threshold was determined to be 20 relative fluorescence units (RFU) based on the NTC. 16 partitions were counted as positive, including 2 positive partitions for cattle-specific Bacteroidales, 3 positive partitions for swine-specific Bacteroidales, 2 positive partitions for human-specific Bacteroidales, and 9 positive partitions for poultry-specific Bacteroidales (FIG. 16). Due to the low copy number of host-specific Bacteroidales, the present data did not support definitive statements about microbial source tracking.

### Certain Definitions

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise.

When ranges are used herein, all combinations and sub-combinations of ranges and specific embodiments therein are intended to be included.

Additionally, the term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “comprising” (and related terms such as “comprise” or “comprises” or “having” or “including”) is not intended to exclude that in other certain embodiments, for example, an embodiment of any compound, composition of matter, composition, method, or process, or the like, described herein, may “consist of” or “consist essentially of” the described features. The term “substantially” can allow for a degree of variability in a value or range, for example, within 90%, within 95%, or within 99% of a stated value or of a stated limit of a range.

Additionally, in describing representative embodiments, a method and/or process may have been presented as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

Further, all publications and patents mentioned herein are incorporated by reference in their entireties for all purposes. In the event of inconsistent usages between this document and those documents so incorporated by reference, the usage in the incorporated reference should be considered supplementary to that of this document; for irreconcilable inconsistencies, the usage in this document controls. Full citations of the references cited herein are provided below.

1. A loop-mediated isothermal amplification (LAMP) assay comprising at least one LAMP primer set that targets a deoxyribonucleic acid (DNA) fragment of fecal indicator bacteria (FIB) in a sample, wherein the assay allows for single-step identification of the presence or absence of the FIB in the sample.

2. The LAMP assay of claim 1, wherein the presence of FIB is indicative of the presence of a foodborne pathogen in the sample, and the absence of FIB is indicative of the absence of a foodborne pathogen in the sample.

3. The LAMP assay of claim 1, wherein the DNA fragment of FIB comprises a 16S rRNA gene sequence and/or the FIB is Bacteroidales, Escherichia coli, and/or Enterococcus faecalis.

4-5. (canceled)

6. The LAMP assay of claim 1, wherein the at least one primer set comprises one or more primers of SEQ ID NO: 4 and SEQ ID NO: 5, one or more primers of SEQ ID NO: 6 and SEQ ID NO: 7, or one or more primers of SEQ ID NO: 8 and SEQ ID NO: 9.

7-8. (canceled)

9. The LAMP assay of claim 1, wherein the at least one primer set comprises primers of SEQ ID NOS: 4-9.

10. The LAMP assay of claim 1, wherein the assay can process and provide a visual result in 60 minutes or less, the visual result indicative of the presence or absence of the FIB in the sample.

11. (canceled)

12. The LAMP assay of claim 1, wherein the at least one LAMP primer set is coupled with a colorimetric reagent.

13. (canceled)

14. The LAMP assay of claim 12, further comprising a fluorescent indicator.

15. The LAMP assay of claim 1, wherein the targeted DNA fragment comprises a species-specific gene.

16. The LAMP assay of claim 1, wherein each of the LAMP primer sets has a limit of detection (LoD) of:
at least about 20 copies/cm² surface area of a collection surface from which the sample was obtained;
at least about 17 copies of FIB/cm² surface area of a collection surface from which the sample was obtained; or
at least about 10³-10⁴ copies/cm² surface area of a collection surface from which the sample was obtained.

17-18. (canceled)

19. A kit comprising:
at least one LAMP primer set of claim 1;
at least one swab for obtaining the sample; and
a heating element to initiate amplification of the targeted DNA fragment when the at least one LAMP primer set and the sample are combined.

20. The kit of claim 19 further comprising:
a fluorescent indicator; and
a fluorescent reader, an ultraviolet light reader, or a camera to provide colorimetric result data indicative of the presence or absence of FIB in the sample.

21. The kit of claim 19, further comprising one or more containers with a reaction mixture therein.

22-25. (canceled)

26. The kit of claim 19, further comprising a plurality of collection flags for the collection of bioaerosol samples, each collection flag comprising a film affixed to a support at a distance away from an end of the support such that, in use, the support can anchor the film a distance above a surface of an area in which the support is positioned.

27-28. (canceled)

29. The kit of claim 26, wherein an LoD of the LAMP primer set is about 17 copies of the FIB per cm² of surface area of the film.

30. A method of monitoring fecal contamination comprising:
providing at least one LAMP primer set of claim 1;
obtaining a sample from a target;
combining the sample and the at least one LAMP primer set into a mixture;
heating the combination to initiate amplification of the targeted DNA fragment; and
detecting a visual result in the heated combination indicative of the presence or absence of the targeted FIB in the sample;
wherein detection of a visual result indicative of the presence of the targeted FIB in the sample is also indicative of the presence of a foodborne pathogen in the sample, and the absence of FIB is indicative of the absence of a foodborne pathogen in the sample.

31. The method of claim 30, wherein the FIB is Bacteroidales and the at least one LAMP primer set comprises primers of SEQ ID NOS. 4-9.

32. (canceled)

33. The method of claim 30, wherein the target comprises a planted field prior to harvest or an unplanted field prior to the growing season, and the sample comprises a plurality of samples collected from various locations across the field.

34. (canceled)

35. The method of claim 33, wherein:
if the presence of the targeted FIB is detected in the sample, further comprising destroying a crop planted in the field, planting crops in the field that are not for human raw consumption, and/or treating the field to remediate any fecal contamination; or
if the absence of the targeted FIB is detected in the sample, further comprising harvesting the crop planted in the field, or planting a crop in the field.

36. (canceled)

37. The method of claim 35, wherein if the presence of the targeted FIB is detected in the sample, further comprising performing a microbial source tracking method.

38-53. (canceled)
